SEQUENCE LISTING (4 sequences)

SEQ ID NO: 1 - protein, 153 aa, Artificial Sequence
  Synthetic polypeptide
MRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 2 - protein, 448 aa, Artificial Sequence
  Synthetic polypeptide
QVQLVESGGGVVQPGRSLRLSCAASGFTFSVYGMNWVRQAPGKGLEWVAIIWYDGDNQYYADSVKGRFTISRDNSKNTLYLQMNGLRAEDTAVYYCARDLRTGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, Artificial Sequence
  Synthetic polypeptide
EIVLTQSPDFQSVTPKEKVTITCRASQSIGSSLHWYQQKPDQSPKLLIKYASQSFSGVPSRFSGSGSGTDFTLTINSLEAEDAAAYYCHQSSSLPFTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 880 aa, Artificial Sequence
  Synthetic polypeptide
SERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVEKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNSGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2024/0199749 A1
(43) Pub. Date: Jun. 20, 2024

(54) Pharmaceutical Composition

(71) Applicant: MedinCell S.A., Jacou (FR)

(72) Inventors: Fang Liu, Jacou (FR); Sylvestre Grizot, Jacou (FR)

(21) Appl. No.: 18/283,350

(22) PCT Filed: Mar. 23, 2022
(86) PCT No.: PCT/EP2022/057684

§ 371 Date: Sep. 21, 2023

(30) Foreign Application Priority Data
Mar. 25, 2021 (GB) 2104224.7

(51) Int. Cl. C07K 16/28; A61K 9/00; A61K 9/16; A61K 39/00; A61K 47/12; A61K 47/20; A61K 47/22; A61P 19/02

(52) U.S. Cl. CPC C07K 16/2866; A61K 9/0019; A61K 9/1611; A61K 9/1623; A61K 9/1647; A61K 47/12; A61K 47/20; A61K 47/22; A61P 19/02; A61K 2039/505

(57) ABSTRACT

The present invention provides a pharmaceutical composition comprising
(a) a triblock copolymer having the formula:

Aᵥ-B_{w}-Aₓ
wherein A is a polyester, B is polyethylene glycol, v and x are the number of repeat units ranging from 1 to 3,000 and w is the number of repeat units ranging from 3 to 300 and v=x or v≠x in an amount of from about 3 to 25 w/w % of the total composition;
(b) a diblock copolymer having the formula:

C_{y}-A_{z}
Wherein A is a polyester, C is an end-capped polyethylene glycol and y and z are the number of repeat units with y ranging from 2 to 250 and z ranging from 1 to 3,000 in an amount of from about 3 to 35 w/w % of the total composition;
(c) a therapeutic protein which is an Interleukin-1 antagonist in an amount of from about 0.5 to 25 w/w % of the total composition;
(d) optionally one or more stabilizer compounds in an amount of from about 0.25 to 15 w/w % of the total composition; and
(e) organic solvent in an amount of from about 50 to 80 w/w % of the total composition.

### FIELD OF THE INVENTION

The present invention relates to sustained release pharmaceutical compositions suitable for intra-articular or peri-articular administration of therapeutic proteins, in particular pharmaceutical compositions for use in preventing, treating or ameliorating rheumatic diseases such as osteoarthritis or crystal arthritis.

### BACKGROUND OF THE INVENTION

Intra-articular (IA) administration of therapeutic agents has been used for several years to prevent pain in osteoarthritis (OA) joints. Arthritis is a common condition causing pain and inflammation in a joint. Osteoarthritis, a degenerative joint disease, and rheumatoid arthritis, an autoimmune form of arthritis, are the two most common types of arthritis. OA prevalence continues to increase over the world.

However, achieving an appropriate IA exposure remains a major challenge as free drugs injected in the IA spaces are rapidly cleared over a period not exceeding a few hours, resulting in poor and insufficient drug levels. (Progress in intra-articular therapy. Evans C. H. et al. (2013) Nat. Rev. Rheumatol; Biomaterial strategies for improved intra-articular drug delivery. Mancipe Castro et al. (2020) Journal of Biomedical Materials Research). As synovial fluid is an ultrafiltrate of the serum, the protein synovial fluid concentration will be a fraction of the serum one, and the serum to synovial fluid protein concentration ratio will vary depending on the protein molecular weight. Typically, for a 150 kDa monoclonal antibody, the synovial fluid concentration will be 4 to 6 fold lower than the serum concentration. However, as already underlined, when injected intra-articularly, a protein will be cleared quickly from the joint thanks to efficient lymphatic drainage and will be retrieved in the systemic compartment. There is thus a need for formulations enabling a sustained release of therapeutic molecules in the IA compartment.

WO199901114, WO2006039704, WO2012019009, WO2014153384, U.S. Pat. Nos. 8,956,636 BB or 8,591,935, WO2020227353 are publications exemplifying formulations composed of poly(lactic-co-glycolic acid) (PLGA) microparticles for the local IA delivery of small molecules such as a corticosteroid (fluticasone, prednisolone, triamcinolone acetonide) or non-steroidal anti-inflammatory drug (celecoxib, ketorolac, ketoprofen, suprofen, tepoxalin) or a combination thereof.

Other biomaterials were evaluated for the IA delivery of such compounds: hyaluronic acid hydrogels, PEG-PLA based in situ forming depots, as described in WO2017085561, or PEG-PCLA hydrogels (Sustained intra-articular release of celecoxib from in situ forming gels made of acetyl-capped PCLA-PEG-PCLA triblock copolymers in horses. Petit A. et al., (2015) Biomaterials).

The US Food and Drug Administration (FDA) recently approved ZILRETTA®, the first and only extended release IA therapy for osteoarthritis-related knee pain (NDA 208845, more information available on https://flexiontherapeutics.com/our-product/ or in Effects of single intra-articular injection of a microsphere formulation of triamcinolone acetonide on knee osteoarthritis pain. Conaghan P G et al. (2018); J Bone JT Surg.).

While these sustained release formulations aim at reducing pain in patients suffering from arthritis, these strategies still present limited long-term benefits and do not prevent arthritis progression. There is thus a need to develop formulations with other types of molecules and in particular with disease modifying osteoarthritis drugs (DMOADS).

Among the DMOADS candidates, a number of biologics have been identified for which stability and functionality within formulations can be challenging. A recent phase II clinical trial highlighted an increase of cartilage thickness after intra articular injections of sprifermin. However, no significant impact on pain was observed which demonstrates the need to improve local drug exposure. (Intra-articular sprifermin reduces cartilage loss in addition to increasing cartilage gain independent of location in the femorotibial joint: post-hoc analysis of a randomised, placebo-controlled phase II clinical trial. Eckstein et al. (2020) Annals of the Rheumatic diseases; Long term efficacy and safety of intra-articular sprifermin in patients with knee osteoarthritis: results from the 5-year forward study. Eckstein et al. (2020) Osteoarthritis and cartilage).

Leconet et al. describe a way to formulate a bispecific antibody within a polymeric PEG-PLA based formulation (Anti-PSMA/CD3 bispecific antibody delivery and antitumor activity using a polymeric depot formulation. Leconet et al. (2018) Molecular Cancer Therapeutics). However, the illustrated formulations were injected subcutaneously and protein functionality or improved bioavailability after IA injection could not be predicted.

There is thus a need to develop new sustained release formulations containing proteins, particularly sustained release formulations comprising therapeutic proteins suitable for intra-articular or peri-articular administration.

### STATEMENT OF INVENTION

An aspect of the invention provides a pharmaceutical composition comprising
(a) a triblock copolymer having the formula:

Aᵥ-B_{w}-Aₓ
wherein A is a polyester, B is polyethylene glycol, v and x are the number of repeat units ranging from 1 to 3,000 and w is the number of repeat units ranging from 3 to 300 and v=x or v≠x in an amount of from about 3 to 25 w/w % of the total composition;
(b) a diblock copolymer having the formula:

C_{y}-A_{z}

Wherein A is a polyester, C is an end-capped polyethylene glycol and y and z are the number of repeat units with y ranging from 2 to 250 and z ranging from 1 to 3,000 in an amount of from about 3 to 35 w/w % of the total composition;
(c) a therapeutic protein which is an Interleukin-1 antagonist in an amount of from about 0.5 to 25 w/w % of the total composition;
(d) optionally one or more stabilizer compounds in an amount of from about 0.25 to 15 w/w % of the total composition; and
(e) organic solvent in an amount of from about 50 to 80 w/w % of the total composition.

The inventors have surprisingly found that the above composition is suitable for the sustained release of therapeutic proteins. The compositions of the invention are particularly suitable for the sustained release of therapeutic proteins which have been administered via intra-articular or peri-articular injection. As discussed above, when injected intra-articularly, typically a protein will be cleared quickly from the joint thanks to efficient lymphatic drainage. By contrast, because the compositions of the invention form a depot (a localized mass formed by precipitation of the pharmaceutical composition) after injection into the joint or in the vicinity of the joint (for intra-articular or peri-articular administration), the rate of protein clearance is significantly reduced, and sustained release of the protein active over a period of weeks or months can be achieved. As well as providing sustained and controlled release of protein therapeutics, the triblock and diblock copolymers degrade and their components are resorbed, avoiding any remaining deposit in the joint after the therapeutic agent has been released. The polymers can be tailored to provide both the appropriate release profile of the therapeutic agent, and controlled degradation and resorption of the polymer vehicle.

In an embodiment of the invention, for the triblock copolymer w is an integer from about 20 to 75 and v and x are each an integer from about 35 to 85; and/or wherein for the triblock copolymer the molecular weight of the PEG is from about 1 to 3 kDa and the polyester repeat unit to ethylene oxide molar ratio is from about 2 to 6.

In an embodiment of the invention, for the triblock copolymer w is an integer from about 20 to 25 and v and x are each an integer from 40 to 85, preferably wherein w is about 23 and v and x are each about 68 or wherein w is an integer from about 40 to 50 and v and x are each an integer from about 35 to 75, preferably wherein w is about 45 and v and x are each about 45 or wherein w is an integer from about 60 to 75 and v and x are each an integer from about 55 to 85, preferably wherein w is about 68 and v and x are each about 68; and/or wherein for the triblock copolymer the molecular weight of the PEG is about 3 kDa and the polyester repeat unit to ethylene oxide molar ratio is about 2; or the molecular weight of the PEG is about 1 kDa and the polyester repeat unit to ethylene oxide molar ratio is about 6 or the molecular weight of the PEG is about 2 kDa and the polyester repeat unit to ethylene oxide molar ratio is about 2.

In an embodiment of the invention, for the diblock copolymer y is an integer from about 20 to 50 and z is an integer from about 75 to 150; and/or wherein for the diblock copolymer the molecular weight of the PEG is from about 1 to 2 kDa and the polyester repeat unit to ethylene oxide molar ratio is from about 2 to 4.

In an embodiment of the invention, for the diblock copolymer y is an integer from about 20 to 25 and z is an integer from about 75 to 110, preferably wherein y is about 23 and z is about 90; or wherein y is an integer from about 40 to 50 and z is an integer from about 100 to 150, preferably wherein y is about 45 and z is about 136; and/or wherein for the diblock copolymer the molecular weight of the PEG is about 1 kDa and the polyester repeat unit to ethylene oxide molar ratio is about 4; or the molecular weight of the PEG is about 2 kDa and the polyester repeat unit to ethylene oxide molar ratio is about 2 or about 3.

The polyester repeat unit is the monomer(s) of the polyester. For example, for poly(lactic acid), the polyester repeat unit is lactic acid, and the relevant molar ratio is the lactic acid/ethylene oxide (LA/EO) molar ratio. For poly(lactic-co-glycolic acid) the monomers are lactic acid and glycolic acid and the relevant molar ratio is the (lactic acid+glycolic acid)/ethylene oxide ((LA+GA)/EO) molar ratio.

Typically, the polyester A for the diblock copolymer is selected from the group of poly(lactic acid), poly(lactic-co-glycolic acid), polyglycolic acid, polycaprolactone, poly(ε-caprolactone-co-lactide), polyethylene adipate, polydioxanone, polyhydroxyalkanoate and mixtures thereof.

Typically, each polyester A for the triblock copolymer is selected from the group of poly(lactic acid), poly(lactic-co-glycolic acid), polyglycolic acid, polycaprolactone, poly(ε-caprolactone-co-lactide), polyethylene adipate, polydioxanone, polyhydroxyalkanoate and mixtures thereof.

In preferred embodiments, the polyester A for the diblock copolymer comprises poly(lactic acid), preferably poly(D,L-lactic acid). More preferably, the polyester is selected from poly(D,L-lactic-co-glycolic acid) or poly(D,L-lactic acid).

In preferred embodiments, each polyester A for the triblock copolymer comprises poly(lactic acid), preferably poly(D,L-lactic acid). More preferably, each polyester is selected from poly(D,L-lactic-co-glycolic acid) or poly(D,L-lactic acid).

Typically, the poly(D,L-lactic-co-glycolic acid) comprises at least 60% (mol/mol) lactic acid, or at least 80% (mol/mol) lactic acid.

A preferred embodiment of the invention provides a pharmaceutical composition comprising
(a) a triblock copolymer having the formula:

PLAᵥ-PEG_{w}-PLAₓ
wherein v and x are the number of repeat units ranging from 1 to 3,000 and w is the number of repeat units ranging from 3 to 300 and v=x or vex in an amount of from about 3 to 25 w/w % of the total composition;
(b) a diblock copolymer having the formula:

mPEG_{y}-PLA_{z}
wherein y and z are the number of repeat units with y ranging from 2 to 250 and z ranging from 1 to 3,000 in an amount of from about 3 to 25 w/w % of the total composition;
(c) a therapeutic protein which is an Interleukin-1 antagonist in an amount of from about 0.5 to 25 w/w % of the total composition;
(d) optionally one or more stabilizer compounds in an amount of from about 0.25 to 15 w/w % of the total composition; and
(e) organic solvent in an amount of from about 50 to 80 w/w % of the total composition.

In an alternative embodiment of the invention provides a pharmaceutical composition comprising:
(a) a triblock copolymer having the formula:

PLGAᵥ-PEG_{w}-PLGAₓ
wherein v and x are the number of repeat units ranging from 1 to 3,000 and w is the number of repeat units ranging from 3 to 300 and v=x or vex in an amount of from about 3 to 25 w/w % of the total composition;
(b) a diblock copolymer having the formula:

mPEG_{y}-PLGA_{z}
wherein y and z are the number of repeat units with y ranging from 2 to 250 and z ranging from 1 to 3,000 in an amount of from about 3 to 25 w/w % of the total composition;
(c) a therapeutic protein which is an Interleukin-1 antagonist in an amount of from about 0.5 to 25 w/w % of the total composition;
(d) optionally one or more stabilizer compounds in an amount of from about 0.25 to 15 w/w % of the total composition; and
(e) organic solvent in an amount of from about 50 to 80 w/w % of the total composition.

In a preferred embodiment, for the triblock copolymer w is an integer from 20 to 25 and v and x are each an integer from 40 to 85, preferably wherein w is about 23 and v and x are each about 68 or wherein w is an integer from 40 to 50 and v and x are each an integer from 35 to 75, preferably wherein w is about 45 and v and x are each about 45 or wherein w is an integer from 60 to 75 and v and x are each an integer from 55 to 85, preferably wherein w is about 68 and v and x are each about 68; and/or wherein for the triblock copolymer the molecular weight of the PEG is about 3 kDa and the lactic acid/ethylene oxide molar ratio is about 2; or the molecular weight of the PEG is 1 kDa and the lactic acid/ethylene oxide molar ratio is about 6 or the molecular weight of the PEG is about 2 kDa and the lactic acid/ethylene oxide molar ratio is about 2.

In a preferred embodiment, for the diblock copolymer y is an integer from 20 to 25 and z is an integer from 75 to 110, preferably wherein y is about 23 and z is about 90; or wherein y is an integer from 40 to 50 and z is an integer from 100 to 150, preferably wherein y is about 45 and z is about 136; and/or wherein for the diblock copolymer the molecular weight of the PEG is about 1 kDa and the lactic acid/ethylene oxide molar ratio is about 4; or the molecular weight of the PEG is about 2 kDa and the lactic acid/ethylene oxide molar ratio is about 2 or about 3.

In a particularly preferred embodiment the therapeutic protein is IL-1Ra, or an amino acid sequence having at least 80%, optionally at least 85%, optionally at least 90%, optionally at least 95%, optionally at least 99% sequence identity to the amino acid sequence according to SEQ ID NO: 1, optionally wherein the therapeutic protein consists of the amino acid sequence according to SEQ ID NO: 1.

(SEQ ID NO: 1)
MRPSGRKSSKMQAFRIWDVNQKTFYLRNNQLVAGYLQGPNVNLEE
KIDVVPIEPHALFLGIHGGKMCLSCVKSGDETRLQLEAVNITDLS
ENRKQDKRFAFIRSDSGPTTSFESAACPGWFLCTAMEADQPVSLT
NMPDEGVMVTKFYFQEDE

In another preferred embodiment the therapeutic protein is canakinumab, optionally an antibody wherein each heavy chain has at least 80%, optionally at least 85%, optionally at least 90%, optionally at least 95%, optionally at least 99% sequence identity to the amino acid sequence according to SEQ ID NO: 2, optionally consisting of SEQ ID NO: 2; and each light chain has at least 80%, optionally at least 85%, optionally at least 90%, optionally at least 95%, optionally at least 99% sequence identity to the amino acid sequence according to SEQ ID NO: 3, optionally consisting of SEQ ID NO: 3.

Heavy chain of canakinumab:
(SEQ ID NO: 2)
QVQLVESGGGVVQPGRSLRLSCAASGFTFSVYGMNWVRQAPGKGL
EWVAIIWYDGDNQYYADSVKGRFTISRDNSKNTLYLQMNGLRAED
TAVYYCARDLRTGPFDYWGQGTLVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKRVEPKSCDKTH
TCPPCPAPELLGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSREEMT
KNQVSLTCLVKGFYPSDIAVEWESNGQPENNYKTTPPVLDSDGSF
FLYSKLTVDKSRWQQGNVFSCSVMHEALHNHYTQKSLSLSPGK
Light chain of canakinumab:
(SEQ ID NO: 3)
EIVLTQSPDFQSVTPKEKVTITCRASQSIGSSLHWYQQKPDQSPK
LLIKYASQSFSGVPSRFSGSGSGTDFTLTINSLEAEDAAAYYCHQ
SSSLPFTFGPGTKVDIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC

In a further preferred embodiment the therapeutic protein is rilonacept, or an amino acid sequence having at least 80%, optionally at least 85%, optionally at least 90%, optionally at least 95%, optionally at least 99% sequence identity to the amino acid sequence according to SEQ ID NO: 4, optionally wherein the therapeutic protein consists of the amino acid sequence according to SEQ ID NO: 4.

Rilonacept:
(SEQ ID NO: 4)
SERCDDWGLDTMRQIQVFEDEPARIKCPLFEHFLKFNYSTAHSAG
LTLIWYWTRQDRDLEEPINFRLPENRISKEKDVLWFRPTLLNDTG
NYTCMLRNTTYCSKVAFPLEVVQKDSCFNSPMKLPVHKLYIEYGI
QRITCPNVDGYFPSSVKPTITWYMGCYKIQNFNNVIPEGMNLSFL
IALISNNGNYTCVVTYPENGRTFHLTRTLTVKVVGSPKNAVPPVI
HSPNDHVVYEKEPGEELLIPCTVYFSFLMDSRNEVWWTIDGKKPD
DITIDVTINESISHSRTEDETRTQILSIKKVTSEDLKRSYVCHAR
SAKGEVAKAAKVKQKVPAPRYTVEKCKEREEKIILVSSANEIDVR
PCPLNPNEHKGTITWYKDDSKTPVSTEQASRIHQHKEKLWFVPAK
VEDSGHYYCVVRNSSYCLRIKISAKFVENEPNLCYNAQAIFKQKL
PVAGDGGLVCPYMEFFKNENNELPKLQWYKDCKPLLLDNIHFSGV
KDRLIVMNVAEKHRGNYTCHASYTYLGKQYPITRVIEFITLEENK
PTRPVIVSPANETMEVDLGSQIQLICNVTGQLSDIAYWKWNGSVI
DEDDPVLGEDYYSVENPANKRRSTLITVLNISEIESRFYKHPFTC
FAKNTHGIDAAYIQLIYPVTNSGDKTHTCPPCPAPELLGGPSVFL
FPPKPKDTLMISRTPEVTCVVVDVSHEDPEVKFNWYVDGVEVHNA
KTKPREEQYNSTYRVVSVLTVLHQDWLNGKEYKCKVSNKALPAPI
EKTISKAKGQPREPQVYTLPPSRDELTKNQVSLTCLVKGFYPSDI
AVEWESNGQPENNYKTTPPVLDSDGSFFLYSKLTVDKSRWQQGNV
FSCSVMHEALHNHYTQKSLSLSPGK.

In a further embodiment the therapeutic protein is a single domain antibody.

The therapeutic protein may have a molecular weight of from about 10 kDa to about 260 kDa, optionally about 10 to about 150 kDa, optionally about 10 to about 146 kDa, optionally about 10 to about 140 kDa, optionally about 10 kDa to about 100 kDa, optionally about 10 kDa to about 50 kDa, optionally about 10 kDa to about 30 kDa, optionally about 15 to about 20 kDa. In preferred embodiments the therapeutic protein may have a molecular weight of from about 10 kDa to about 30 kDa, optionally from about 15 to about 20 kDa.

The composition may comprise from about 3 to 20 w/w % of the therapeutic protein, optionally from about 4 to 16 w/w % of the therapeutic protein.

The composition may comprise from about 60 to 80 w/w %, optionally from about 65 to 80 w/w %, preferably from about 65 to 75 w/w % organic solvent. The organic solvent may be selected from benzyl alcohol, benzyl benzoate, dimethyl isosorbide (DMI), ethyl acetate, ethyl benzoate, ethyl lactate, glycerol formal, methyl ethyl ketone, methyl isobutyl ketone, N-ethyl-2-pyrrolidone, pyrrolidone-2, tetraglycol, triacetin, tributyrin, tripropionin, glycofurol, and mixtures thereof. Preferably the organic solvent is tripropionin or benzyl benzoate.

The composition may comprise about 1.5 to 10 w/w % stabilizer compound, optionally about 4 to 8 w/w % stabilizer compound, optionally about 5 w/w %.

In preferred embodiments the stabilizer compound is trehalose, L-methionine, sucrose, mannitol, ascorbic acid, arginine, polysorbate 80, polysorbate 20 or a combination thereof, preferably trehalose, L-methionine or a combination thereof.

In preferred embodiments the composition comprises from about 3 to about 20 w/w %, optionally from about 3 to about 15 w/w %, optionally from about 3 to 10 w/w %, optionally from about 9 w/w % of the triblock copolymer.

In preferred embodiments the composition comprises from about 3 to about 25 w/w %, optionally from about 3 to about 20 w/w %, optionally from about 3 to about 15 w/w %, optionally from about 3 to 10 w/w %, optionally from about 9 w/w % of the diblock copolymer.

In preferred embodiment, the composition comprises less than about 50 w/w % total copolymer, preferably less than about 40 w/w % total copolymer.

The composition may further comprise one or more salts and/or one or more buffering agents, optionally wherein the buffering agent is sodium phosphate or histidine.

A further aspect of the invention provides a composition as defined above for use in medicine.

An additional aspect of the invention provides a composition as defined above for use in preventing, treating or ameliorating rheumatic disease in a subject. The rheumatic disease may be selected from osteoarthritis, crystal arthritis, post-traumatic osteoarthritis (PTOA), rheumatoid arthritis, ankylosing spondylitis, fibromyalgia, infectious arthritis, juvenile idiopathic arthritis, lupus erythematosus, polymyalgia rheumatica, psoriatic arthritis, reactive arthritis and scleroderma, preferably osteoarthritis, PTOA or crystal arthritis.

In a preferred embodiment the use comprises administering the composition to the subject by intra-articular injection or peri-articular injection. Intra-articular is a particularly preferred mode of administration. The composition may be injected into or in the vicinity of a synovial joint such as a knee, ankle, elbow, humerus, ulna, pivot joint, ball and socket joint, hinge joint, shoulder, hip, scapula, leg joint, fibula, saddle joint, wrist joint, finger joint, toe joint or tibia, preferably knees, toe, shoulder or hip.

The composition or composition for use of the invention is suitable for forming a depot when injected into the body.

In a further aspect provided is a method of preventing, treating or ameliorating rheumatic disease in a subject, the method comprising the step of administering a composition as defined above to the subject. The rheumatic disease may be selected from osteoarthritis, crystal arthritis, post-traumatic osteoarthritis (PTOA), rheumatoid arthritis, ankylosing spondylitis, fibromyalgia, infectious arthritis, juvenile idiopathic arthritis, lupus erythematosus, polymyalgia rheumatica, psoriatic arthritis, reactive arthritis and scleroderma, preferably osteoarthritis, PTOA or crystal arthritis.

In a preferred embodiment the composition is administered to the subject by intra-articular injection or peri-articular injection. Intra-articular injection is a particularly preferred mode of administration. The composition may be injected into or in the vicinity of a synovial joint such as a knee, ankle, elbow, humerus, ulna, pivot joint, ball and socket joint, hinge joint, shoulder, hip, scapula, leg joint, fibula, saddle joint, wrist joint, finger joint, toe joint or tibia, preferably knees, toe, shoulder or hip.

In one embodiment the composition is administered using an 18G to 26G needle, optionally a 21G to 23G needle.

In preferred embodiments of the composition for use or method of the invention, the ratio of the concentration of the therapeutic protein in the synovial fluid to the concentration of the therapeutic protein in the serum of the subject is greater than 10, optionally greater than 50, optionally greater than 100 for a period of at least 7 days, optionally 14 days, optionally 21 days, optionally 28 days, optionally 1 month, optionally 2 months, optionally 3 months after administration of the composition.

In a further aspect, provided is a method of preparing a pharmaceutical composition, the method comprising
a) preparing an aqueous solution of a therapeutic protein as defined above;
b) optionally adding a stabilizer compound as defined above to the solution;
c) spray drying the solution of step b) to form a spray-dried product;
d) dissolving a triblock copolymer as defined above and a diblock copolymer as defined above in an organic solvent as defined above;
e) adding the spray-dried product to the polymer composition formed in step d) and mixing the composition to form a dispersion of the therapeutic protein.

Typically the amount of triblock and diblock copolymers dissolved in step d) is sufficient to provide a final dispersion in step e) comprising the triblock copolymer in an amount of from 3 to 25 w/w % of the final composition and the diblock copolymer in an amount of from 3 to 25 w/w %.

Typically the amount of therapeutic protein in the dispersion of step e) is from 0.5 to 25 w/w %, optionally from about 3 to 20 w/w %, optionally from about 4 to 16 w/w %.

Typically the amount of stabilizer compound in the final dispersion is from about 0.25 to 15 w/w %, optionally about 1.5 to 10 w/w % stabilizer compound, optionally about 4 to 8 w/w % stabilizer compound, optionally about 5 w/w %.

The aqueous solution of step a) may comprise one or more salts and/or one or more buffering agents. The aqueous solution may comprise sodium phosphate or histidine. The aqueous solution may comprise polysorbate 80 or L-methionine.

In one embodiment the product of step b) is filtered prior to spray drying.

### BRIEF DESCRIPTION OF THE DRAWINGS

FIG. 1 shows the synovial fluid concentration profiles of IL-1Ra up to 28 days after IA injection of 3 different formulations F1, F2 and F3 and a control saline solution. Formulations were prepared as detailed in Example 2 and a pharmacokinetics study was performed as explained in Example 3. Data evidence sustained release of the protein from the 3 candidate formulations in synovial fluid and modulation of the release profiles depending on the formulation compositions.

FIG. 2 shows the percentage of IL-1Ra retrieved from knees and depots grinding samples 1, 3, 7, 14 and 28 days after formulations IA injection. Formulations were prepared as detailed in Example 2 and pharmacokinetics study was performed as explained in Example 3.

FIG. 3 illustrates the evolution up to 37 days after rat treatment of delta knee swelling calculated as the difference between control left knee and operated right knee circumferences. Formulations were prepared as detailed in Example 2 and pharmacodynamic study was performed as explained in Example 4. Data show a decrease of delta with time, suggesting that all test items injected were well tolerated and did not induce a further knee swelling.

FIG. 4 shows the evolution up to 37 days after rat treatment of weight bearing deficit, calculated as the difference between the weight applied on the left hind control paw and the operated and treated right hind paw in gram. Formulations were prepared as detailed in Example 2 and pharmacodynamic study was performed as explained in Example 4. Data show that 5 weeks after treatment, formulations F4 and F5 present similar results to the positive control, suggesting an efficacity of the tested compositions.

FIG. 5 shows the evolution of D-Lactic acid content quantified in knees of rats euthanized 1, 21, 42 and 84 days after intra-articular administration of different polymeric vehicles. Test items were prepared as detailed in Example 2 and the IA injection and data treatment were performed as explained in Example 5. A decrease of D-Lactic acid content is observed at the end of the study and suggests a resorption of the copolymers within the intra-articular area.

FIG. 6 illustrates the qualitative evolution of PEG content visualized by immunohistochemistry (IHC) within the IA area of rats 1, 21, 42 and 84 days after administration of different polymeric vehicles. Test items were prepared as detailed in Example 2 and the IA injection and data treatment were performed as explained in Example 5. The decrease of the PEG immunohistochemistry scoring after several weeks indicates a lower presence of PEG within the knee sample sections which is in line with the polyester resorption observed in FIG. 5.

### DETAILED DESCRIPTION OF THE INVENTION

An aspect of the invention provides a pharmaceutical composition comprising:
(a) a triblock copolymer having the formula:

Aᵥ-B_{w}-Aₓ
wherein A is a polyester, B is polyethylene glycol, v and x are the number of repeat units ranging from 1 to 3,000 and w is the number of repeat units ranging from 3 to 300 and v=x or v≠x in an amount of from about 3 to 25 w/w % of the total composition;
(b) a diblock copolymer having the formula:

C_{y}-A_{z}

Wherein A is a polyester, C is an end-capped polyethylene glycol and y and z are the number of repeat units with y ranging from 2 to 250 and z ranging from 1 to 3,000 in an amount of from about 3 to 35 w/w % of the total composition;
(c) a therapeutic protein which is an Interleukin-1 antagonist in an amount of from about 0.5 to 25 w/w % of the total composition;
(d) optionally one or more stabilizer compounds in an amount of from about 0.25 to 15 w/w % of the total composition; and
(e) organic solvent in an amount of from about 50 to 80 w/w % of the total composition.

The polyester repeat unit is the monomer(s) of the polyester. For example, for poly(lactic acid), the polyester repeat unit is lactic acid, and the relevant molar ratio is the lactic acid/ethylene oxide (LA/EO) molar ratio. For poly(lactic-co-glycolic acid) the monomers are lactic acid and glycolic acid and the relevant molar ratio is the (lactic acid+glycolic acid)/ethylene oxide ((LA+GA)/EO) molar ratio.

In an embodiment of the invention, for the triblock copolymer w is an integer from about 20 to 75 and v and x are each an integer from about 35 to 85; and/or wherein for the triblock copolymer the molecular weight of the PEG is from about 1 to 3 kDa and the polyester repeat unit to ethylene oxide molar ratio is from about 2 to 6.

In an embodiment of the invention, for the triblock copolymer w is an integer from about 20 to 25 and v and x are each an integer from 40 to 85, preferably wherein w is about 23 and v and x are each about 68 or wherein w is an integer from about 40 to 50 and v and x are each an integer from about 35 to 75, preferably wherein w is about 45 and v and x are each about 45 or wherein w is an integer from about 60 to 75 and v and x are each an integer from about 55 to 85, preferably wherein w is about 68 and v and x are each about 68; and/or wherein for the triblock copolymer the molecular weight of the PEG is about 3 kDa and the polyester repeat unit to ethylene oxide molar ratio is about 2; or the molecular weight of the PEG is about 1 kDa and the polyester repeat unit to ethylene oxide molar ratio is about 6 or the molecular weight of the PEG is about 2 kDa and the polyester repeat unit to ethylene oxide molar ratio is about 2.

In an embodiment of the invention, for the diblock copolymer y is an integer from about 20 to 50 and z is an integer from about 75 to 150; and/or wherein for the diblock copolymer the molecular weight of the PEG is from about 1 to 2 kDa and the polyester repeat unit to ethylene oxide molar ratio is from about 2 to 4.

In an embodiment of the invention, for the diblock copolymer y is an integer from about 20 to 25 and z is an integer from about 75 to 110, preferably wherein y is about 23 and z is about 90; or wherein y is an integer from about 40 to 50 and z is an integer from about 100 to 150, preferably wherein y is about 45 and z is about 136; and/or wherein for the diblock copolymer the molecular weight of the PEG is about 1 kDa and the polyester repeat unit to ethylene oxide molar ratio is about 4; or the molecular weight of the PEG is about 2 kDa and the polyester repeat unit to ethylene oxide molar ratio is about 2 or about 3.

In an embodiment of the invention for the triblock copolymer w is an integer from about 20 to 75 and v and x are each an integer from about 35 to 85; and/or wherein for the triblock copolymer the molecular weight of the PEG is from about 1 to 3 kDa and the polyester repeat unit to ethylene oxide molar ratio is from about 2 to 6; and for the diblock copolymer y is an integer from about 20 to 50 and z is an integer from about 75 to 150; and/or wherein for the diblock copolymer the molecular weight of the PEG is from about 1 to 2 kDa and the polyester repeat unit to ethylene oxide molar ratio is from about 2 to 4.

Typically, the polyester A for the diblock copolymer is selected from the group of poly(lactic acid), poly(lactic-co-glycolic acid), polyglycolic acid, polycaprolactone, poly(ε-caprolactone-co-lactide), polyethylene adipate, polydioxanone, polyhydroxyalkanoate and mixtures thereof.

Typically, each polyester A for the triblock copolymer is selected from the group of poly(lactic acid), poly(lactic-co-glycolic acid), polyglycolic acid, polycaprolactone, poly(ε-caprolactone-co-lactide), polyethylene adipate, polydioxanone, polyhydroxyalkanoate and mixtures thereof.

In preferred embodiments, the polyester A for the diblock copolymer comprises poly(lactic acid), preferably poly(D,L-lactic acid). More preferably, the polyester is selected from poly(D,L-lactic-co-glycolic acid) or poly(D,L-lactic acid).

In preferred embodiments, each polyester A for the triblock copolymer comprises poly(lactic acid), preferably poly(D,L-lactic acid). More preferably, each polyester is selected from poly(D,L-lactic-co-glycolic acid) or poly(D,L-lactic acid).

Typically, the poly(D,L-lactic-co-glycolic acid) comprises at least 60% (mol/mol) lactic acid, or at least 80% (mol/mol) lactic acid.

A preferred embodiment of the invention provides a pharmaceutical composition comprising:
(a) a triblock copolymer having the formula:

PLAᵥ-PEG_{w}-PLAₓ
wherein v and x are the number of repeat units ranging from 1 to 3,000 and w is the number of repeat units ranging from 3 to 300 and v=x or vex in an amount of from about 3 to 25 w/w % of the total composition;
(b) a diblock copolymer having the formula:

mPEG_{y}-PLA_{z}
wherein y and z are the number of repeat units with y ranging from 2 to 250 and z ranging from 1 to 3,000 in an amount of from about 3 to 25 w/w % of the total composition;
(c) a therapeutic protein which is an Interleukin-1 antagonist in an amount of from about 0.5 to 25 w/w % of the total composition;
(d) optionally one or more stabilizer compounds in an amount of from about 0.25 to 15 w/w % of the total composition; and
(e) organic solvent in an amount of from about 50 to 80 w/w % of the total composition.

In an alternative embodiment of the invention provides a pharmaceutical composition comprising:
(a) a triblock copolymer having the formula:

PLGAᵥ-PEG_{w}-PLGAₓ
wherein v and x are the number of repeat units ranging from 1 to 3,000 and w is the number of repeat units ranging from 3 to 300 and v=x or vex in an amount of from about 3 to 25 w/w % of the total composition;
(b) a diblock copolymer having the formula:

mPEG_{y}-PLGA_{z}
wherein y and z are the number of repeat units with y ranging from 2 to 250 and z ranging from 1 to 3,000 in an amount of from about 3 to 25 w/w % of the total composition;
(c) a therapeutic protein which is an Interleukin-1 antagonist in an amount of from about 0.5 to 25 w/w % of the total composition;
(d) optionally one or more stabilizer compounds in an amount of from about 0.25 to 15 w/w % of the total composition; and
(e) organic solvent in an amount of from about 50 to 80 w/w % of the total composition.

The composition described above is typically suitable for forming a depot when injected into the body, i.e. an “in situ depot”.

The compositions of the invention are administered via depot injection. The term “depot injection” is an injection of a flowing pharmaceutical composition that deposits a drug in a localized mass, such as a solid or semi-solid mass, called a “depot”. The depots as defined herein are in situ forming upon injection. Thus, the formulations can be prepared as solutions or suspensions and can be injected into the body. In the present invention the composition is typically administered via intra-articular or peri-articular injection and a depot is formed in or in the vicinity of a joint.

An “in situ depot” is a solid or semi-solid, localized mass formed by precipitation of the pharmaceutical composition after injection of the composition into the subject. The pharmaceutical composition comprises copolymers which are substantially insoluble in aqueous solution. Thus, when the pharmaceutical composition contacts the aqueous environment of the human or animal body, a phase inversion occurs causing the composition to change from a liquid to a solid, i.e. precipitation of the composition occurs, leading to formation of an “in situ depot”.

An “in situ depot” can be clearly distinguished from hydrogel pharmaceutical formulations described in the prior art. Hydrogels have three-dimensional networks that are able to absorb large quantities of water. The polymers making up hydrogels are soluble in aqueous solution. By contrast, the polymers used in the present invention are substantially insoluble in aqueous solution. The pharmaceutical compositions of the invention are free of water, or substantially free of water. For example, the pharmaceutical compositions of the invention comprise less than 1.5% w/w water, optionally less than 1.2% w/w water.

The triblock and diblock copolymers used in the present invention are “bioresorbable” which means that the block copolymers undergo hydrolytic cleavage in vivo to form their constituent (m)PEG and oligomers or monomers derived from the polyester block. For example, PLA undergoes hydrolysis to form lactic acid. The result of the hydrolysis process leads to a progressive mass loss of the depot, i.e. to a depot resorption, and ultimately to its disappearance.

As used herein “repeat units” are the fundamental recurring units of a polymer.

As used herein “polyethylene glycol”, as abbreviated PEG throughout the application, is sometimes referred to as poly(ethylene oxide) or poly(oxyethylene) and the terms are used interchangeably in the present invention. By “end-capped polyethylene glycol” (cPEG) refers to PEG's in which one terminal hydroxyl group is reacted and includes alkoxy-capped PEG's, urethane-capped PEG's, ester-capped PEG's and like compounds. The capping group is a chemical group which does not contain a chemical function susceptible to react with cyclic esters like lactide, glycolactide, caprolactone and the like or other esters and mixtures thereof. For example, the reaction of an end-capped PEG polymer with lactide generates a diblock cPEG-PLA copolymer. The end-capped PEG is preferably methoxy polyethylene glycol (mPEG).

The abbreviation of “PLA” refers to poly(lactic acid). PLA is typically poly(D,L-lactic acid), also referred to as poly-D,L-lactide, poly[oxy(1-methyl-2-oxoethylene)], or poly[3,6-dimethyl-1,4-dioxane-2,5-dione].

The abbreviation of “PLGA” refers to poly(lactic-co-glycolic acid). PLGA is typically poly(D,L lactic-co-glycolic acid), also referred to as poly(D,L-lactide-co-glycolide) or a 1,4-dioxane-2,5-dione, 3,6-dimethyl-, polymer with 1,4-dioxane-2,5-dione.

The copolymers have been named as follows:

The PLA-PEG-PLA triblock copolymers described herein are labelled PaRb where a represents the molecular weight of the PEG chain in kDa and b is the lactic acid/ethylene oxide (LA/EO) molar ratio and allows the calculation of the PLA chain length within the copolymer.

The PLGA-PEG-PLGA triblock copolymers described herein are labelled DLKG-PaRb. DLKG-PaRb stands for a linear PLGA-PEG-PLGA triblock copolymer composed of D,L-lactide (DL) and glycolide (G) and where k is the molar ratio (%) of LA compared to GA; a represents the molecular weight of the PEG chain in kDa and b is the (lactic acid+glycolic acid)/ethylene oxide ((LA+GA)/EO) molar ratio. For example, DL80G-P2R2 is a triblock PLGA-PEG-PLGA with a 2 kDA PEG block, an overall [(LA+GA)/EO] molar ratio of 2 and wherein each polyester arm is composed of 80% lactic acid.

The mPEG-PLA diblock copolymers described herein are labelled dPaRb where a represents the molecular weight of the mPEG chain in kDa and b is the lactic acid/ethylene oxide (LA/EO) molar ratio.

The mPEG-PLGA diblock copolymers described herein are labelled DLKG-dPaRb. DLKG-dPaRb stands for a linear mPEG-PLGA diblock copolymer composed of D,L-lactide (DL) and glycolide (G) and where k is the molar ratio of LA compared to GA; a represents the molecular weight of the PEG chain in kDa and b is the (lactic acid+glycolic acid)/ethylene oxide ((LA+GA)/EO) molar ratio. For example, DL80G-dP2R2.4 is a mPEG-PLGA diblock copolymer with a 2 kDa PEG block, an overall [(LA+GA)/EO] molar ratio of 2.4 and wherein each polyester arm is composed of 80% lactic acid.

The molecular weight of the PEG, also referred to as the PEG chain or PEG repeat unit, namely —(CH₂CH₂O)ₙ— where n is an integer, is measured by gel permeation chromatography (GPC), typically with a polystyrene standard. The molecular weight measured is number average molecular weight (Mn).

Similarly, the molecular weight of the entire diblock copolymer or triblock copolymer is typically measured by gel permeation chromatography (GPC), usually with a polystyrene standard. The molecular weight measured is number average molecular weight (Mn).

General formulae for the mPEG-PLA diblock and PLA-PEG-PLA triblock copolymers are set out below:

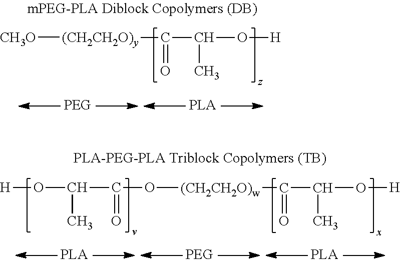

A PLA-PEG-PLA copolymer may be obtainable by ring-opening polymerization of lactide by PEG. A mPEG-PLA diblock copolymer may be obtainable by ring-opening polymerization of lactide by methoxyPEG. If diblock and/or triblock copolymers comprising PLGA are required, glycolide is added together with DL-lactide to reach the targeted (LA+GA)/EO molar ratio.

In one embodiment, the lactide is preferably D,L-lactide, leading to the formation of polymers comprising poly-D,L-lactide (PDLLA), i.e. the triblock copolymer may be PDLLAv-PEGw-PDLLAx and the diblock copolymer may be mPEGy-PDLLAz

General formulae for the mPEG-PLGA diblock and PLGA-PEG-PLGA triblock copolymers are set out below:

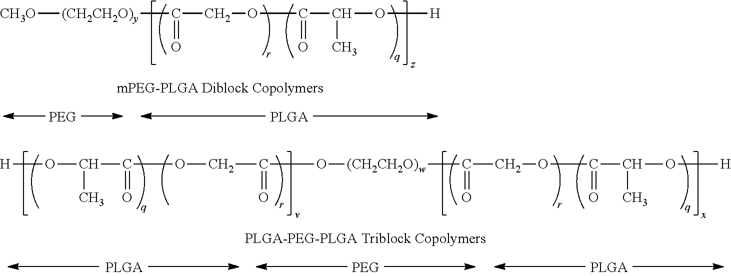

The LA/GA molar ratio k can be defined as q/r and the polyester repeat unit (z for the diblock copolymer and x and v for the triblock copolymer) is the sum of q+r. k is typically at least 60% (mol/mol) (i.e. at least 60% of lactic acid within the PLGA polyester), or at least 80% (mol/mol).

The compositions of the invention comprise a therapeutic protein which is an Interleukin-1 antagonist. The therapeutic protein may be an antagonist of IL-1α or IL-1β. An agonist is a chemical that binds to a receptor and activates the receptor to produce a biological response, for example Interleukin-1. In contrast, an antagonist blocks the action of the agonist.

The Interleukin-1 (IL-1) family is a group of 11 cytokines, which induce a complex network of proinflammatory cytokines and via expression of integrins on leukocytes and endothelial cells, regulates and initiates inflammatory responses. IL-1α and IL-1β are the most studied members, because they were discovered first and because they possess strongly proinflammatory effect. They have a natural antagonist IL-1Ra (IL-1 receptor antagonist). All three of them include a beta trefoil fold and bind IL-1 receptor (IL-1R). However, in contrast to IL-1α and IL-1β whose binding to the IL-1 receptor will promote the recruitment of the IL-1 receptor accessory protein (IL-1RAcP) and further signalling via MyD88 adaptor, IL-1Ra binding to IL-1R will prevent the recruitment of IL-1RAcP. IL-1Ra regulates IL-1α and IL-1β proinflammatory activity by competing with them for binding sites of the receptor.

Nine IL-1 superfamily members occur in a single cluster on human chromosome two; sequence and chromosomal anatomy evidence suggest these formed through a series of gene duplications of a proto-IL-1β ligand. In this way, IL-1β, IL-1α, IL-36α, IL-36β, IL-36γ, IL-36RA, IL-37, IL-38, and IL-1RA are very likely ancestral family members sharing a common lineage. However, IL-18 and IL-33 are on different chromosomes and there is insufficient sequence or chromosomal anatomy evidence to suggest they share common ancestry with the other IL-1 superfamily members. IL-33 and IL-18 have been included into the IL-1 superfamily due to structural similarities, overlap in function and the receptors involved in their signalling.

In a particularly preferred embodiment, the therapeutic protein is Interleukin-1 receptor antagonist (IL-1Ra).

In a particularly preferred embodiment, the therapeutic protein is IL-1Ra, or an amino acid sequence having at least 80%, optionally at least 85%, optionally at least 90%, optionally at least 95%, optionally at least 99% sequence identity to the amino acid sequence according to SEQ ID NO: 1, optionally wherein the therapeutic protein consists of the amino acid sequence according to SEQ ID NO: 1.

(SEQ ID NO: 1)
MRPSGRKSSKMQAFRIWDVNQKTFYLRNNQLVAGYLQGPNVNLEE
KIDVVPIEPHALFLGIHGGKMCLSCVKSGDETRLQLEAVNITDLS
ENRKQDKRFAFIRSDSGPTTSFESAACPGWFLCTAMEADQPVSLT
NMPDEGVMVTKFYFQEDE,

SEQ ID NO: 1 is the amino acid sequence of a modified form of the Human interleukin-1 receptor antagonist (IL-1Ra), also known as Anakinra and commercially available as the product Kineret® from Sobi, Inc, produced in Escherichia coli cells by recombinant DNA technology. Anakinra is a protein that differs from the sequence of the human Interleukin-1 receptor antagonist by the addition of one methionine at its N-terminus; it also differs from the human protein in that it is not glycosylated, as it is manufactured in Escherichia coli.

IL-1Ra functions as a competitive inhibitor of the IL-1 receptor in vivo and in vitro. It counteracts the effects of both IL-1α and IL-1β. Upon binding of IL-1Ra, the IL-1 receptor does not transmit a signal to the cell. IL-1Ra inhibits the release of both IL-1α and IL-1β, IL-2 secretion, cell surface IL-2 receptor expression. It blocks the stimulation of prostaglandin E2 synthesis in synovial cells and thymocyte proliferation. It also inhibits the release of leukotriene B4 from monocytes after stimulation with bacterial lipopolysaccharides. It blocks insulin release from isolated pancreatic cells.

In another preferred embodiment the therapeutic protein is canakinumab, optionally an antibody wherein each heavy chain has at least 80%, optionally at least 85%, optionally at least 90%, optionally at least 95%, optionally at least 99% sequence identity to the amino acid sequence according to SEQ ID NO: 2, optionally consisting of SEQ ID NO: 2; and each light chain has at least 80%, optionally at least 85%, optionally at least 90%, optionally at least 95%, optionally at least 99% sequence identity to the amino acid sequence according to SEQ ID NO: 3, optionally consisting of SEQ ID NO: 3.

Heavy chain of canakinumab:
(SEQ ID NO: 2)
QVQLVESGGGVVQPGRSLRLSCAASGFTFSVYGMNWVRQAPGKGL
EWVAIIWYDGDNQYYADSVKGRFTISRDNSKNTLYLQMNGLRAED
TAVYYCARDLRTGPFDYWGQGTLVTVSSASTKGPSVFPLAPSSKS
TSGGTAALGCLVKDYFPEPVTVSWNSGALTSGVHTFPAVLQSSGL
YSLSSVVTVPSSSLGTQTYICNVNHKPSNTKVDKRVEPKSCDKTH
TCPPCPAPELLGGPSVFLFPPKPKDTLMISRTPEVTCVVVDVSHE
DPEVKFNWYVDGVEVHNAKTKPREEQYNSTYRVVSVLTVLHQDWL
NGKEYKCKVSNKALPAPIEKTISKAKGQPREPQVYTLPPSREEMT
KNQVSLTCLVKGFYPSDIAVEWESNGQPENNYKTTPPVLDSDGSF
FLYSKLTVDKSRWQQGNVFSCSVMHEALHNHYTQKSLSLSPGK
Light chain of canakinumab:
(SEQ ID NO: 3)
EIVLTQSPDFQSVTPKEKVTITCRASQSIGSSLHWYQQKPDQSPK
LLIKYASQSFSGVPSRFSGSGSGTDFTLTINSLEAEDAAAYYCHQ
SSSLPFTFGPGTKVDIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSFNRGEC.

Canakinumab, also known as ACZ885, is a recombinant, human anti-human-IL-1β monoclonal antibody that belongs to the IgG1/κ isotype subclass. Canakinumab is a monoclonal antibody comprising two heavy chains and two light chains. It is expressed in a murine Sp2/0-Ag14 cell line and comprised of two 448 residue heavy chains and two 214-residue light chains, with a molecular mass of 145157 Daltons when deglycosylated. Both heavy chains of canakinumab contain oligosaccharide chains linked to the protein backbone at asparagine 298 (Asn 298). Canakinumab binds to human IL-1β and neutralizes its inflammatory activity by blocking its interaction with IL-1 receptors, but it does not bind IL-1α or IL-1Ra. Canakinumab is commercially available under the brand name Ilaris® from Novartis.

In a further preferred embodiment the therapeutic protein is rilonacept, or an amino acid sequence having at least 80%, optionally at least 85%, optionally at least 90%, optionally at least 95%, optionally at least 99% sequence identity to the amino acid sequence according to SEQ ID NO: 4, optionally wherein the therapeutic protein consists of the amino acid sequence according to SEQ ID NO: 4.

Rilonacept:
(SEQ ID NO: 4)
SERCDDWGLDTMRQIQVFEDEPARIKCPLFEHFLKFNYSTAHSAG
LTLIWYWTRQDRDLEEPINFRLPENRISKEKDVLWFRPTLLNDTG
NYTCMLRNTTYCSKVAFPLEVVQKDSCFNSPMKLPVHKLYIEYGI
QRITCPNVDGYFPSSVKPTITWYMGCYKIQNFNNVIPEGMNLSFL
IALISNNGNYTCVVTYPENGRTFHLTRTLTVKVVGSPKNAVPPVI
HSPNDHVVYEKEPGEELLIPCTVYFSFLMDSRNEVWWTIDGKKPD
DITIDVTINESISHSRTEDETRTQILSIKKVTSEDLKRSYVCHAR
SAKGEVAKAAKVKQKVPAPRYTVEKCKEREEKIILVSSANEIDVR
PCPLNPNEHKGTITWYKDDSKTPVSTEQASRIHQHKEKLWFVPAK
VEDSGHYYCVVRNSSYCLRIKISAKFVENEPNLCYNAQAIFKQKL
PVAGDGGLVCPYMEFFKNENNELPKLQWYKDCKPLLLDNIHFSGV
KDRLIVMNVAEKHRGNYTCHASYTYLGKQYPITRVIEFITLEENK
PTRPVIVSPANETMEVDLGSQIQLICNVTGQLSDIAYWKWNGSVI
DEDDPVLGEDYYSVENPANKRRSTLITVLNISEIESRFYKHPFTC
FAKNTHGIDAAYIQLIYPVTNSGDKTHTCPPCPAPELLGGPSVFL
FPPKPKDTLMISRTPEVTCVVVDVSHEDPEVKFNWYVDGVEVHNA
KTKPREEQYNSTYRVVSVLTVLHQDWLNGKEYKCKVSNKALPAPI
EKTISKAKGQPREPQVYTLPPSRDELTKNQVSLTCLVKGFYPSDI
AVEWESNGQPENNYKTTPPVLDSDGSFFLYSKLTVDKSRWQQGNV
FSCSVMHEALHNHYTQKSLSLSPGK.

Rilonacept is a dimeric fusion protein consisting of the ligand-binding domains of the extracellular portions of the human interleukin-1 receptor (IL-1R1) and IL-1 receptor accessory protein (IL-1RAcP) linked in-line to the fragment-crystallizable portion (Fc region) of human lgG1 that binds and neutralizes IL-1. It has a molecular weight of approximately 251 kDa. Rilonacept is available commercially as ARCALYST® from Regeneron.

In a further embodiment the therapeutic protein is a single domain antibody. These were originally developed from Camelid antibodies which are single chain antibodies devoid of a light chain.

A single-domain antibody (sdAb), also known as a nanobody, is an antibody fragment consisting of only the variable domain of the heavy chain of the antibody. Like a whole antibody, it is able to bind selectively to a specific antigen. With a molecular weight of about 12-15 kDa, single-domain antibodies are much smaller than common antibodies (150-160 kDa) which are composed of two heavy protein chains and two light chains, and even smaller than Fab fragments (about 50 kDa, one light chain and half a heavy chain) and single-chain variable fragments (about 25 kDa, two variable domains, one from a light and one from a heavy chain).

As used herein, “sequence identity” is determined by comparing the sequence of the reference amino acid sequence to that portion of another amino acid sequence so aligned so as to maximize overlap between the two sequences while minimizing sequence gaps, wherein any overhanging sequences between the two sequences are ignored.

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A .; Devereux et al. (1984) Nucleic Acids Res. 12: 387). Examples of other software that can perform sequence comparisons include, but are not limited to, the BLAST package (see Ausubel et al. (1999) ibid-Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174:247-50; FEMS Microbiol. Lett. (1999) 177:187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

Variant sequences with a particular level of sequence identity to SEQ ID NOs: 1 to 4 can be been modified in such a manner that the polypeptide in question substantially retains its function. A variant sequence can be obtained by addition, deletion, substitution, modification, replacement and/or variation of at least one residue present in reference sequence.

Typically, amino acid substitutions may be made, for example from 1, 2 or 3 to 10 or 20 substitutions provided that the modified sequence substantially retains the required activity or function. Amino acid substitutions may include the use of non-naturally occurring analogues.

Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R H |
| AROMATIC | | F W Y |

The term “homology” may be equated with the term “identity”.

Preferably, reference to a sequence which has a percent identity to any one of the SEQ ID NOs detailed herein refers to a sequence which has the stated percent identity over the entire length of the SEQ ID NO referred to.

The therapeutic protein may have a molecular weight of from about 10 kDa to about 260 kDa, optionally about 10 to about 150 kDa, optionally about 10 to about 146 kDa, optionally about 10 to about 140 kDa, optionally about 10 kDa to about 100 kDa, optionally about 10 kDa to about 50 kDa, optionally about 10 kDa to about 30 kDa, optionally about 15 to about 20 kDa. In preferred embodiments the therapeutic protein may have a molecular weight of from about 10 kDa to about 30 kDa, optionally from about 15 to about 20 kDa.

The molecular weight of the protein can be determined from the amino acid sequence, or can be measured by a method known to a person skilled in the art, for example MALDI mass spectrometry or electrospray ionisation mass spectrometry.

The composition may comprise from about 3 to 20 w/w % of the therapeutic protein, optionally from about 4 to 16 w/w % of the therapeutic protein.

The composition may comprise from about 60 to 80 w/w %, optionally from about 65 to 80 w/w %, preferably from about 65 to 75 w/w % organic solvent. The organic solvent may be selected from benzyl alcohol, benzyl benzoate, dimethyl isosorbide (DMI), ethyl acetate, ethyl benzoate, ethyl lactate, glycerol formal, methyl ethyl ketone, methyl isobutyl ketone, N-ethyl-2-pyrrolidone, pyrrolidone-2, tetraglycol, triacetin, tributyrin, tripropionin, glycofurol, and mixtures thereof. Preferably the organic solvent is tripropionin or benzyl benzoate.

The composition may comprise about 1.5 to 10 w/w % stabilizer compound, optionally about 4 to 8 w/w % stabilizer compound, optionally about 5 w/w %. The stabilizer compound is not especially limited provided it stabilizes the therapeutic protein in aqueous solution, providing an improvement when spray drying the protein to form a powder or cake. The stabilizer may prevent formation of high molecular weight (HMW) species and/or oxidation of the protein and/or provide improved aerosol formation and/or particle size or form of the final spray-dried powder.

The stabilizer compound may prevent aggregation and/or denaturation of the therapeutic protein. For example, trehalose is a stabilizer compound which will protect protein during the drying process.

The stabilizer compound may prevent oxidation of the therapeutic protein, particularly oxidation of methionine residues. L-methionine is a stabilizer compound which prevents oxidation of methionine residues in the therapeutic protein by providing an alternative reactant for the oxidation reaction.

In preferred embodiments the stabilizer compound is trehalose, L-methionine, sucrose, mannitol, ascorbic acid, arginine, polysorbate 80 (also referred to as Tween® 80), polysorbate 20 (also referred to as Tween® 20) or a combination thereof, preferably trehalose, L-methionine or a combination thereof.

The naturally occurring isomer of trehalose is α,α-Trehalose or α-D-glucopyranosyl-(1→1)-α-D-glucopyranoside:

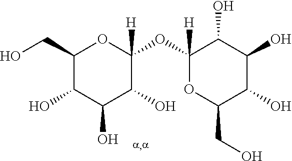 [SMILES: OC[C@H]1O[C@H](O[C@H]2O[C@H](CO)[C@@H](O)[C@H](O)[C@H]2O)[C@H](O)[C@@H](O)[C@@H]1O]

In preferred embodiments the composition comprises from about 3 to about 20 w/w %, optionally from about 3 to about 15 w/w %, optionally from about 3 to 10 w/w %, optionally from about 9 w/w % of the triblock copolymer.

In preferred embodiments the composition comprises from about 3 to about 20 w/w %, optionally from about 3 to about 15 w/w %, optionally from about 3 to 10 w/w %, optionally from about 9 w/w % of the diblock copolymer.

In preferred embodiment, the composition comprises less than about 50 w/w % total copolymer, preferably less than about 40 w/w % total copolymer.

The composition or composition for use may further comprise one or more pharmaceutically-acceptable excipients.

The composition may further comprise one or more salts and/or one or more buffering agents, optionally wherein the buffering agent is sodium phosphate or histidine.

The buffering agent maintains the aqueous solution containing the protein within a particular pH range.

The composition may comprise one or more of citric acid, sodium chloride, disodium edetate dihydrate, Polysorbate 80 and sodium hydroxide.

The “injectability” of a formulation, as used herein, is defined by the force needed in Newtons (N) to inject a formulation using pre-determined parameters. These parameters include injection speed, injection volume, injection duration, syringe type or needle type and the like. These parameters may vary based on specific formulation, or the desired method of administration such as peri-neural, intra articular and the like. They may be adjusted to be able to observe the differences and fluctuations between the formulations. The injectability must be kept low such that the formulation can be easily administered by a qualified healthcare professional in an acceptable timeframe. An acceptable injectability value may be from 0.1 N to 20N. A non-optimal injectability may be greater than 20 N but less than 30 N. Injectability may be measured using a texturometer, preferably a Lloyd Instruments FT plus texturometer, using the following analytical conditions: 500 μL of formulation are injected through a 1 ml syringe, using a 23G 1″ Terumo needle with a 1 or 2 mL/min flow rate at room temperature.

“Viscosity,” by definition and as used herein, is a measure of a fluid's resistance to flow and gradual deformation by shear stress or tensile strength. It describes the internal friction of a moving fluid. For liquids, it corresponds to the informal concept of “thickness”. By ‘dynamic viscosity” is meant a measure of the resistance to flow of a fluid under an applied force. The dynamic velocity can range from 10 mPa·s to 3000 mPa·s. Preferably the composition has a dynamic viscosity of from about 500 to about 2000 mPa·s, optionally from about 500 to about 1000 mPa·s.

Dynamic viscosity of the vehicles used to make the final formulations are determined using an Anton Paar Rheometer equipped with cone plate measuring system. Typically, 700 μL of studied vehicle are placed on the measuring plate. The temperature is controlled at+25° C. The measuring system used is a cone plate with a diameter of 50 mm and a cone angle of 1 degree (CP50-1). The working range is from 10 to 1000 s⁻¹, preferably 100 s⁻¹. Vehicles are placed at the center of the thermo-regulated measuring plate using a positive displacement pipette. The measuring system is lowered down and a 0.104 mm gap is left between the measuring system and the measuring plate. 21 viscosity measurement points are determined across the 10 to 1000 s⁻¹ shear rate range. Given values are the ones obtained at 100 s⁻¹.

A further aspect of the invention provides a composition as defined above for use in medicine.

An additional aspect of the invention provides a composition as defined above for use in preventing, treating or ameliorating rheumatic disease in a subject. Rheumatic diseases are autoimmune and inflammatory diseases that cause the immune system to attack joints, muscles, bones, and organs.

The rheumatic disease may be selected from osteoarthritis, crystal arthritis, post-traumatic osteoarthritis (PTOA), rheumatoid arthritis, ankylosing spondylitis, fibromyalgia, infectious arthritis, juvenile idiopathic arthritis, lupus erythematosus, polymyalgia rheumatica, psoriatic arthritis, reactive arthritis,) and scleroderma, preferably osteoarthritis, PTOA or crystal arthritis.

Osteoarthritis is the most common type of joint disease, affecting more than 20 million individuals in the United States alone. It is a painful, degenerative joint disease that often involves the hips, knees, neck, lower back, or small joints of the hands. Osteoarthritis usually develops in joints that are injured by repeated overuse from performing a particular task or playing a favorite sport or from carrying around excess body weight.

Osteoarthritis can be thought of as a degenerative disorder arising from the biochemical breakdown of articular (hyaline) cartilage in the synovial joints. However, the current view holds that osteoarthritis involves not only the articular cartilage but the entire joint organ, including the subchondral bone and synovium.

Crystal arthritis is a joint disorder that is characterized by accumulation of tiny crystals in one or more joints. Crystal arthritis includes gout caused by the formation of uric acid crystals and pseudogout, also called Calcium pyrophosphate dihydrate (CPPD) crystal deposition disease, caused by the formation of calcium pyrophosphate crystals.

Post-traumatic osteoarthritis, also referred to as post-traumatic arthritis, is a joint disease similar to osteoarthritis but consecutive a joint injury or trauma.

In a preferred embodiment the use comprises administering the composition to the subject by intra-articular injection or peri-articular injection. Intra-articular injection is a particularly preferred mode of administration. When the therapeutic protein is IL1-Ra or related sequences peri-articular administration may be preferred.

As used herein, “intra-articular” refers to the space inside of a joint between two bones, specifically to the portion of the joint contained by the joint capsule. Meaning “inside of a joint,” intra-articular may refer to the space itself or, in the case of the body's movable joints, to any tissues or fluid found inside of the synovial membrane, the lining of the joint capsule. Within the synovial membrane is the synovial fluid, the lubricating fluid of the joint, as well as articular cartilage, which provides a near frictionless gliding surface or cushion between the adjoining bony surfaces. Other joint types may feature ligaments in their intra-articular space that hold the two bones together. In synovial or movable joints, these tissues are extra-articular, or outside of the joint capsule.

As used herein “peri-articular” administration means administration in the vicinity of or around a joint.

The composition may be injected into or in the vicinity of a synovial joint such as a knee, ankle, elbow, humerus, ulna, pivot joint, ball and socket joint, hinge joint, shoulder, hip, scapula, leg joint, fibula, saddle joint, wrist joint, finger joint, toe joint or tibia, preferably knee, toe, shoulder or hip.

The composition or composition for use of the invention is suitable for forming a depot when injected into the body.

In a further aspect provided is a method of preventing, treating or ameliorating a rheumatic disease in a subject, the method comprising the step of administering a composition as defined above to the subject. The rheumatic disease may be selected from osteoarthritis, crystal arthritis, post-traumatic osteoarthritis (PTOA), rheumatoid arthritis, ankylosing spondylitis, fibromyalgia, infectious arthritis, juvenile idiopathic arthritis, lupus erythematosus, polymyalgia rheumatica, psoriatic arthritis, reactive arthritisand scleroderma, preferably osteoarthritis, PTOA or crystal arthritis.

In a preferred embodiment the composition is administered to the subject by intra-articular injection or peri-articular injection. Intra-articular injection is a particularly preferred mode of administration. The composition may be injected into or in the vicinity of a synovial joint such as a knee, ankle, elbow, humerus, ulna, pivot joint, ball and socket joint, hinge joint, shoulder, hip, scapula, leg joint, fibula, saddle joint, wrist joint, finger joint, toe joint or tibia, preferably knees, toe, shoulder or hip.

In one embodiment the composition is administered using a 18G to 26G needle, optionally an 21G to 23G needle.

In preferred embodiments of the composition for use or method of the invention, the ratio of the concentration of the therapeutic protein in the synovial fluid to the concentration of the therapeutic protein in the serum of the subject is greater than 10, optionally greater than 50, optionally greater than 100 for a period of at least 7 days, optionally 14 days, optionally 21 days, optionally 28 days, optionally 1 month, optionally 2 months, optionally 3 months after administration of the composition.

In a further aspect, provided is a method of preparing a pharmaceutical composition, the method comprising
a) preparing an aqueous solution of a therapeutic protein as defined above;
b) optionally adding a stabilizer compound as defined above to the solution;
c) spray drying the solution of step b) to form a spray-dried product;
d) dissolving a triblock copolymer as defined above and a diblock copolymer as defined above in an organic solvent as defined above;
e) adding the spray-dried product to the polymer composition formed in step d) and mixing the composition to form a dispersion of the therapeutic protein.

The spray-dried product may be a powder or a cake.

Typically the amount of triblock and diblock copolymers dissolved in step d) is sufficient to provide a final dispersion in step e) comprising the triblock copolymer in an amount of from 3 to 25 w/w % of the final composition and the diblock copolymer in an amount of from 3 to 25 w/w %.

Typically the amount of therapeutic protein in the dispersion of step e) is from 0.5 to 25 w/w %, optionally from about 3 to 20 w/w %, optionally from about 4 to 16 w/w %. The mixing may be achieved in step e) using a magnetic stirrer.

Typically the amount of stabilizer compound in the final dispersion is from about 0.25 to 15 w/w %, optionally about 1.5 to 10 w/w % stabilizer compound, optionally about 4 to 8 w/w % stabilizer compound, optionally about 9 w/w %.

The aqueous solution of step a) may comprise one or more salts and/or one or more buffering agents. The aqueous solution may comprise sodium phosphate or histidine. The aqueous solution may comprise polysorbate 80 or L-methionine.

The aqueous solution may have a pH from about 5.0 to 6.0. A histidine buffer can be used to provide a pH within the range of from about 5.0 to 6.0.

The aqueous solution may be a commercially available solution of the therapeutic composition, optionally mixed with a stabilizer in step b). For example, in one preferred embodiment 50 w/w % trehalose was added to a solution of Kineret®.

Alternatively, the composition of the aqueous solution may be modified by buffer exchange. For example, disposable ultrafiltration centrifugal devices with a polyethersulfone (PES) membrane for concentration, desalting, and buffer exchange of biological samples can be used to provide a final solution composition appropriate for spray drying. Such products may have a molecular weight cutoff (MWCO) of 10 kDa and can be used for processing sample volumes ranging from about 5 to 20 mL, for example Thermo Scientific™ Pierce™ Protein Concentrators PES. For example, in a preferred embodiment the solution composition can be modified by buffer exchange in 10 kDa MWCO PES concentrators using cycles of concentration-dilution in 10 mM sodium phosphate pH 7.0, 4 mg/mL trehalose and 0.02 w/w % Tween 80. In another example, the buffer can be exchanged using PD-10 desalting columns.

In one embodiment the product of step b) is filtered prior to spray drying. The solution may be filtered using regenerated cellulose filter, for example a 0.2 μm regenerated cellulose filter.

The spray drying process may be performed using a spray drying machine for example a mini spray dryer such as the mini spray dryer B-290 from Büchi (Switzerland). Operating conditions for the mini spray dryer B-290 may be set as follows: inlet temperature of 85° C., flow rate of 1.5 mL/min, aspirator rate at 100% and air flow varying between 600-1000 L/h. The observed outlet temperature may be from about 57 to 60° C.

Wherever embodiments are described with the language “comprising,” otherwise analogous embodiments described in terms of “consisting of” and/or “consisting essentially of” are included.

All of the references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

The present invention is also defined with reference to the following clauses:

1. A pharmaceutical composition comprising
(a) a triblock copolymer having the formula:

PLAᵥ-PEG_{w}-PLAₓ
wherein v and x are the number of repeat units ranging from 1 to 3,000 and w is the number of repeat units ranging from 3 to 300 and v=x or v≠x in an amount of from about 3 to 25 w/w % of the total composition;
(b) a diblock copolymer having the formula:

mPEG_{y}-PLA_{z}
wherein y and z are the number of repeat units with y ranging from 2 to 250 and z ranging from 1 to 3,000 in an amount of from about 3 to 25 w/w % of the total composition;
(c) a therapeutic protein which is an Interleukin-1 antagonist in an amount of from about 0.5 to 25 w/w % of the total composition;
(d) optionally one or more stabilizer compounds in an amount of from about 0.25 to 15 w/w % of the total composition; and
(e) organic solvent in an amount of from about 50 to 80 w/w % of the total composition.

2. A pharmaceutical composition according to clause 1 wherein for the triblock copolymer w is an integer from 20 to 25 and v and x are each an integer from 40 to 85, preferably wherein w is about 23 and v and x are each about 68 or wherein w is an integer from 40 to 50 and v and x are each an integer from 35 to 75, preferably wherein w is about 45 and v and x are each about 45 or wherein w is an integer from 60 to 75 and v and x are each an integer from 55 to 85, preferably wherein w is about 68 and v and x are each about 68; and/or wherein for the triblock copolymer the molecular weight of the PEG repeat unit is 3 kDa and the lactic acid/ethylene oxide molar ratio is 2; or the molecular weight of the PEG repeat unit is 1 kDa and the lactic acid/ethylene oxide molar ratio is 6 or the molecular weight of the PEG repeat unit is 2 kDa and the lactic acid/ethylene oxide molar ratio is 2.

3. A composition according to clause 1 or clause 2 wherein for the diblock copolymer y is an integer from 20 to 25 and z is an integer from 75 to 110, preferably wherein y is about 23 and z is about 90; or wherein y is an integer from 40 to 50 and z is an integer from 100 to 150, preferably wherein y is about 45 and z is about 136; and/or wherein for the diblock copolymer the molecular weight of the PEG repeat unit is 1 kDa and the lactic acid/ethylene oxide molar ratio is 4; or the molecular weight of the PEG repeat unit is 2 kDa and the lactic acid/ethylene oxide molar ratio is 3.

4. A composition according to any preceding clause wherein the therapeutic protein is IL-1Ra, or an amino acid sequence having at least 80%, optionally at least 85%, optionally at least 90%, optionally at least 95%, optionally at least 99% sequence identity to the amino acid sequence according to SEQ ID NO: 1, optionally wherein the therapeutic protein consists of the amino acid sequence according to SEQ ID NO: 1.

5. A composition according to any of clauses 1 to 3 wherein the therapeutic protein is canakinumab, optionally an antibody wherein each heavy chain has at least 80%, optionally at least 85%, optionally at least 90%, optionally at least 95%, optionally at least 99% sequence identity to the amino acid sequence according to SEQ ID NO: 2, optionally consisting of SEQ ID NO: 2; and each light chain has at least 80%, optionally at least 85%, optionally at least 90%, optionally at least 95%, optionally at least 99% sequence identity to the amino acid sequence according to SEQ ID NO: 3, optionally consisting of SEQ ID NO: 3.

6. A composition according to any of clauses 1 to 3 wherein the therapeutic protein is rilonacept, or an amino acid sequence having at least 80%, optionally at least 85%, optionally at least 90%, optionally at least 95%, optionally at least 99% sequence identity to the amino acid sequence according to SEQ ID NO: 4, optionally wherein the therapeutic protein consists of the amino acid sequence according to SEQ ID NO: 4.

7. A composition according to any of clauses 1 to 3 wherein the therapeutic protein is a single domain antibody.

8. A composition according to any preceding clause wherein the therapeutic protein has a molecular weight of from about 10 kDa to about 260 kDa, optionally about 10 to about 150 kDa, optionally about 10 to about 146 kDa, optionally about 10 to about 140 kDa, optionally about 10 kDa to about 100 kDa, optionally about 10 kDa to about 50 kDa, optionally about 10 kDa to about 30 kDa, optionally about 15 to about 20 kDa.

9. A composition according to any preceding clause comprising from about 3 to 20 w/w % of the therapeutic protein, optionally from about 4 to 16 w/w % of the therapeutic protein.

10. A composition according to any preceding clause comprising from about 60 to 80 w/w %, optionally from about 65 to 80 w/w %, preferably from about 65 to 75 w/w % organic solvent.

11. A composition according to any preceding clause wherein the organic solvent is selected from benzyl alcohol, benzyl benzoate, dimethyl isosorbide (DMI), ethyl acetate, ethyl benzoate, ethyl lactate, glycerol formal, methyl ethyl ketone, methyl isobutyl ketone, N-ethyl-2-pyrrolidone, pyrrolidone-2, tetraglycol, triacetin, tributyrin, tripropionin, glycofurol, and mixtures thereof.

12. A composition according to clause 11 wherein the organic solvent is tripropionin or benzyl benzoate.

13. A composition according to any preceding clause comprising about 1.5 to 10 w/w % stabilizer compound, optionally about 4 to 8 w/w % stabilizer compound, optionally about 5 w/W %.

14. A composition according to any preceding clause wherein the stabilizer compound is trehalose, L-methionine, sucrose, mannitol, ascorbic acid, arginine, polysorbate 80, polysorbate 20 or a combination thereof, preferably trehalose, L-methionine or a combination thereof.

15. A composition according to any preceding clause comprising from about 3 to about 20 w/w %, optionally from about 3 to about 15 w/w %, optionally from about 3 to 10 w/w %, optionally from about 9 w/w % of the triblock copolymer.

16. A composition according to any preceding clause comprising from about 3 to about 20 w/w %, optionally from about 3 to about 15 w/w %, optionally from about 3 to 10 w/w %, optionally from about 9 w/w % of the diblock copolymer.

17. A composition according to any preceding clause further comprising one or more salts and/or one or more buffering agents, optionally wherein the buffering agent is sodium phosphate or histidine.

18. A composition according to any preceding clause for use in medicine.

19. A composition according to any preceding clause for use in preventing, treating or ameliorating a rheumatic disease in a subject.

20. A composition for use according to clause 19 wherein the rheumatic disease is selected from osteoarthritis, crystal arthritis, post-traumatic arthritis, rheumatoid arthritis, ankylosing sondylitis, fibromyalagia, infectious arthritis, juvenile idiopathic arthritis, lupus erythematosus, polymyalgia rheumatica, psoriatic arthritis, reactive arthritis and sclerodoma, preferably osteoarthritis or crystal arthritis.

21. A composition for use according to any of clauses 18 to 20 wherein the use comprises administering the composition to the subject by intra-articular injection or peri-articular injection.

22. A composition for use according to clause 21 wherein the composition is injected into or in the vicinity of a synovial joint such as a knee, ankle, elbow, humerus, ulna, pivot joint, ball and socket joint, hinge joint, shoulder, hip, scapula, leg joint, fibula, saddle joint, wrist joint, finger joint, toe joint or tibia of the subject, preferably the knee, toe, shoulder or hip.

23. A composition or composition for use according to any preceding clause which is suitable for forming a depot when injected into the body.

24. A method of preventing, treating or ameliorating rheumatic disease in a subject, the method comprising the step of administering a composition as defined in any preceding clause to the subject.

25. A method according to clause 24 wherein the rheumatic disease is selected from osteoarthritis, crystal arthritis, post-traumatic arthritis, rheumatoid arthritis, ankylosing sondylitis, fibromyalagia, infectious arthritis, juvenile idiopathic arthritis, lupus erythematosus, polymyalgia rheumatica, psoriatic arthritis, reactive arthritis and sclerodoma, preferably osteoarthritis or crystal arthritis.

26. A method according to clause 24 or clause 25 wherein the composition is administered to the subject by intra-articular injection or peri-articular injection.

27. A method according to clause 26 wherein the composition is injected into or in the vicinity of a synovial joint such as a knee, ankle, elbow, humerus, ulna, pivot joint, ball and socket joint, hinge joint, shoulder, hip, scapula, leg joint, fibula, saddle joint, wrist joint, finger joint, toe joint or tibia of the subject, preferably the knee, toe, shoulder or hip.

28. A composition for use or method according to any of clauses 18 to 27 wherein the composition is administered using an 18G to 26G needle, optionally a 21G to 23G needle.

29. A composition for use or method according to any of clauses 18 to 28 wherein the ratio of the concentration of the therapeutic protein in the synovial fluid to the concentration of the therapeutic protein in the serum of the subject is greater than 10, optionally greater than 50, optionally greater than 100 for a period of at least 7 days, optionally 14 days, optionally 21 days, optionally 28 days, optionally 1 month, optionally 2 months, optionally 3 months after administration of the composition.

30. A method of preparing a pharmaceutical composition, the method comprising
a) preparing an aqueous solution of a therapeutic protein as defined in any preceding clause;
b) optionally adding a stabilizer compound as defined in any preceding clause to the solution;
c) spray drying the solution of step b) to form a spray-dried product;
d) dissolving a triblock copolymer as defined in any preceding clause and a diblock copolymer as defined in any preceding clause in an organic solvent as defined in any preceding clause;
e) adding the spray-dried product to the polymer composition formed in step d) and mixing the composition to form a dispersion of the therapeutic protein.

31. A method according to clause 30 wherein the amount of triblock and diblock copolymers dissolved in step d) is sufficient to provide a final dispersion in step e) comprising the triblock copolymer in an amount of from 3 to 25 w/w % of the final composition and the diblock copolymer in an amount of from 3 to 25 w/w %.

32. A method according to clause 30 or clause 31 wherein the amount of therapeutic protein in the dispersion of step e) is from 0.5 to 25 w/w %, optionally from about 3 to 20 w/w %, optionally from about 4 to 16 w/w %.

33. A method according to any of clauses 30 to 32 wherein the amount of stabilizer compound in the final dispersion is from about 0.25 to 15 w/w %, optionally about 1.5 to 10 w/w % stabilizer compound, optionally about 4 to 8 w/w % stabilizer compound, optionally about 5 w/w %.

34. A method according to any of clauses 30 to 33 wherein the aqueous solution of step a) comprises one or more salts and/or one or more buffering agents.

35. A method according to clause 34 wherein the aqueous solution comprises sodium phosphate or histidine.

36. A method according to any of clauses 30 to 35 wherein the aqueous solution comprises polysorbate 80.

A method according to any of clauses 30 to 36 wherein the product of step b) is filtered prior to spray drying.

### EXAMPLES

### Example 1: Materials

### Linear Block Copolymers Synthesis

Copolymers were synthesized according to the method described in the U.S. Pat. No. 6,350,812, incorporated herein by reference, with minor modifications. Typically, the necessary amount of PEG (gives the triblock co-polymer) or methoxy-PEG (gives the diblock copolymer) was heated at 65° C. and dried under vacuum for 2 hours in a reactor vessel. D,L-lactide (corresponding to the targeted LA/EO molar ratio) and zinc lactate ( 1/1000 of amount of lactide) were added. If polymers comprising PLGA were required, glycolide was added together with D,L-lactide to reach the targeted (LA+GA)/EO molar ratio. The reaction mixture was first dehydrated by three short vacuum/N2 cycles. The reaction mixture was heated at 140° C. and rapidly degassed under vacuum. The reaction was conducted for four days at 140° C. under constant nitrogen flow (0.2 bar). The reaction was cooled to room temperature and its content was dissolved in acetone and then subjected to precipitation with ethanol. The product obtained was subsequently dried under reduced pressure. Alternatively, the copolymers may be purchased from Corbion.

The triblock polymers described herein are typically labelled PaRb or when applicable DLKGPaRb, where a represents the size of the PEG chain in kDa, b is the (LA+GA)/EO molar ratio and k is the molar fraction of LA compared to GA. The diblock polymers described herein are labelled dPaRb or DLkGdPaRb where a represents the size of the PEG chain in kDa, b is the (LA+GA)/EO molar ratio and k is the molar fraction of LA compared to GA.

The product obtained was characterized by ¹H NMR for its residual lactide content and for the determination of the R ratio. ¹H NMR spectroscopy was performed using a Brucker Advance 300 MHz spectrometer. For all ¹H NMR spectrograms, TopSpin software was used for the integration of peaks and their analyses. Chemical shifts were referenced to the δ=7.26 ppm solvent value of CDCl₃.

For the determination of the R ratio, which describes the ratio between polyester units over ethylene oxide units ((LA+GA)/EO), all peaks were integrated separately. The intensity of the signal (integration value) is directly proportional to the number of hydrogens that constitutes the signal. To determine the R ratio ((LA+GA)/EO ratio), the integration values need to be homogenous and representative of the same number of protons (e.g. all signal values are determined for 1H). One characteristic peak of PLA, one of PLGA and one of PEG are then used to determine the (LA+GA)/EO ratio. This method is valid for molecular weights of PEGs above 1000 g/mol where the signal obtained for the polymer end-functions can be neglected.

### IL-1Ra

Human IL-1Ra protein, more specifically human IL-1Ra with an additional methionine at the N-terminus, SEQ ID NO:1, was obtained as the Kineret® product from Sobi, Inc., a protein solution in ready-to-use syringes at a protein concentration of 150 mg/mL.

### Example 2: IL-1Ra Formulations Preparation

### Spray Drying:

In order to obtain a spray dried cake with an appropriate protein content, trehalose was added to the Kineret® solution to reach typically a 30 to 50% (w/w) trehalose content in the final powder preparation.

Alternatively, solution composition can be modified by buffer exchanged in 10 kDa MWCO PES concentrators using cycles of concentration-dilution in 10 mM sodium phosphate pH 7.0, 4 mg/mL trehalose and 0.02% (w/w) Tween 80.

Different excipients, typically other sugars such as sucrose or free methionine to avoid oxidation events, can be added to the protein solution during the buffer exchange procedure to adjust the spray dried cake final composition. Likewise, a histidine buffer presenting a more acidic pH (5.0 to 6.0 range) can be used as acidic pH can decrease the generation of deamidation events.

The protein final solution was filtered on a 0.2 um regenerated cellulose filter prior to the spray drying process that was performed using the mini spray dryer B-290 from Büchi (Switzerland). Operating conditions were set as follows: inlet temperature of 85° C., flow rate of 1.5 mL/min, aspirator rate at 100% and air flow varying between 600-1000 L/h. The observed outlet temperature during the run was comprised between 57 and 60° C. Protein powder was collected into a sterile glass vial and was further dried overnight in vacuum oven before storage at 4° C. The protein content was assessed by SEC-HPLC and the trehalose content can be measured using an enzymatic kit provided by Libios.

### Formulation Preparation:

A polymeric vehicle was prepared by solubilizing the required amount of copolymers in organic solvent, typically tripropionin or benzyl benzoate, to reach the final polymer content. After full solubilization of copolymers, the required amount of IL-1Ra spray dried cake was dispersed into the vehicle to obtain a protein formulation with the targeted final protein concentration. The formulation was then stirred with a magnetic stirrer for 30 min at room temperature to achieve good powder dispersion and adequate formulation homogeneity.

### Example 3: IL-1Ra Pharmacokinetics Study

A pharmacokinetics study was launched to compare three formulations of IL-1Ra and one control IL-1Ra saline solution (Kineret® solution diluted ×3 in sterile NaCl 0.9%). Candidate formulations were prepared as disclosed in Example 2. Table 1 presents their compositions.

TABLE 1

| Test | IL-1Ra | Trehalose | TB | TB % | DB | DB % | Solvent | Solvent |
|---|---|---|---|---|---|---|---|---|
| item | % (w/w) | % (w/w) | type | (w/w) | type | (w/w) | type | % (w/w) |
| F1 | 5.0 | 4.5 | P3R2 | 9.0 | dP2R3 | 9.0 | Tripropionin | 72.0 |
| F2 | 5.0 | 4.5 | P1R6 | 9.0 | dP1R4 | 9.0 | Tripropionin | 72.0 |
| F3 | 5.0 | 4.5 | P3R2 | 9.0 | dP2R3 | 9.0 | Benzyl benzoate | 72.0 |

Briefly, 2.5 μL of test items were injected intra-articularly into the right knee of ten-week-old C5BL/6J mice using a 10 μL Hamilton syringe with a 26G needle. After 1, 3, 7, 14 and 28 days, 4 mice per group of candidate formulations were euthanized. Blood samples were withdrawn by intra-cardiac puncture and their right knees were recovered and incubated in 1 mL cell culture medium for 1 hour at room temperature. After incubation time, samples were centrifugated and synovial fluids (i.e. supernatants) were collected. The synovial fluid volume of a mouse knee was arbitrarily estimated to be 5 μL so that the protein concentration measured in the supernatants had to be multiply 200 fold to get an estimation of the synovial fluid concentration. Knee joints were further grinded and incubated 1 hour in 1 mL of medium to quantify proteins remaining within the depots. For control group, an additional retro-orbital blood sampling was withdrawn at 4 hours and only one timepoint at day 1 was performed. IL-1Ra was quantified on appropriately diluted solutions of serum and synovial fluid using an Elisa kit developed by Abcam® (reference ab100564).

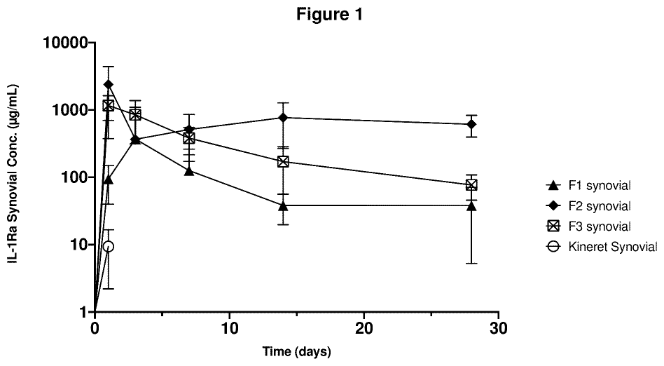

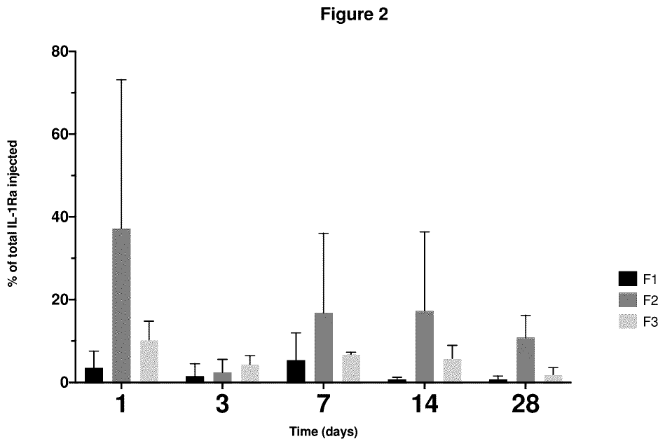

Results of quantification in synovial fluid and grinded knees are presented in FIGS. 1 and 2. Apart from day 1 samples, concentration in the serums were always below 0.5 ng/ml, meaning far below the concentrations measured in synovial fluid. Different release profiles were obtained with the three tested formulations, showing that the modification of formulation composition efficiently modulated the IL-1Ra release within knee articulations. Moreover, the measured synovial concentrations from candidate formulations were higher than the one of control saline IL-1Ra injection and quantifiable up to at least 28 days after injection. Finally, samples from grinded knees show that part of the protein initially injected was still present within the depot at all timepoints. These values tend to decrease with time which is in accordance with a sustained release of the protein in the synovial fluid.

### Example 4: IL-1Ra Pharmacodynamic Study

A pharmacodynamic study aiming at comparing the efficacy of formulations presenting two different IL-1Ra concentrations was performed.

An osteoarthritis (OA) animal model was mechanically generated via the transection of the Anterior Cruciate Ligament (ACL) on the right knee for all the rats fourteen days before test items administration (Day −14).

Briefly, male Wistar rats were anesthetized in an induction chamber using 5% isoflurane and then maintained with 2% isoflurane via a custom-made facemask or with Ketamine/Xylazine mixture. The right joint skin was shaved, and the area was sterilized. An incision was made in the medial aspect of the joint capsule (anterior to medial collateral ligament), the anterior cruciate ligament was transected and using required precautions, joint capsule was sutured. After this procedure, an iodine solution was used to sterilize the wound area and cefazolin (100 mg/kg/day) was intramuscularly administrated for 3 days to prevent infection.

On the day of injection (t0) animals were identified and randomized into 5 groups of 8 animals. Negative control group received no treatment. Positive control received a 500 μL daily intraperitoneal injection of 5 mg/mL IL-1Ra saline solution for five consecutive days during 3 weeks (15 injections representing a total dose of 37.5 mg). Group 3, 4 and 5 were administered to the right knee as a 20 uL single intra-articular injection of vehicle V4, formulation F4 loaded at 4% of IL-1Ra and formulation F5 loaded at 16% of IL-1Ra, respectively. Administrations were performed using a 100 μL Hamilton® syringe equipped with a 25G needle. Table 2 summarizes the different groups.

TABLE 2

| | | | Injec- | | |
|---|---|---|---|---|---|
| | | Adminis- | tion | Injec- | IL-1Ra |
| | | tration | volume | tion | dose |
| Group | Test item | route | (μL) | number | (mg) |
| 1 | Negative control: no | — | — | — | — |
| | treatment | | | | |
| 2 | Positive control: daily | IP | 500 | 15 | 37.5 |
| | injection of IL-1Ra in | | | | |
| | saline solution | | | | |
| 3 | Single injection of V4 | IA | 20 | 1 | — |
| 4 | Single injection of F4 | IA | 20 | 1 | 0.9 |
| 5 | Single injection of F5 | IA | 20 | 1 | 3.6 |

Formulations or vehicle were prepared as disclosed in Example 2 and homogenized before syringe filling. Their compositions are detailed in Table 3.

TABLE 3

| Test | IL-1Ra | Trehalose | P3R2 | dP2R2.4 | |
|---|---|---|---|---|---|
| item | % (w/w) | % (w/w) | % (w/w) | % (w/w) | Tripropionin |
| V4 | — | — | 10.0 | 10.0 | 80.0 |
| F4 | 3.9 | 2.1 | 9.4 | 9.4 | 75.2 |
| F5 | 16.3 | 8.7 | 3.8 | 3.8 | 67.4 |

The efficacy of the test compounds was evaluated after treatment administration by assessing:
clinical signs daily (appearance, behaviour, suffering sign, food/water intake)
knee swelling using a manual caliper and the other knee as a control once a week for up to 6 weeks
pain monitoring using an incapacitance apparatus (Bio-SWB-TOUCH-S, Biosed) measuring weight bearing deficit, once a week for up to 6 weeks.

Furthermore, cartilage degradation was evaluated after animal euthanasia (3 weeks after injection for half of each group, and 6 weeks after injection for remaining animals) through histological analyses of knee joints samples after toluidine blue staining.

### Results:

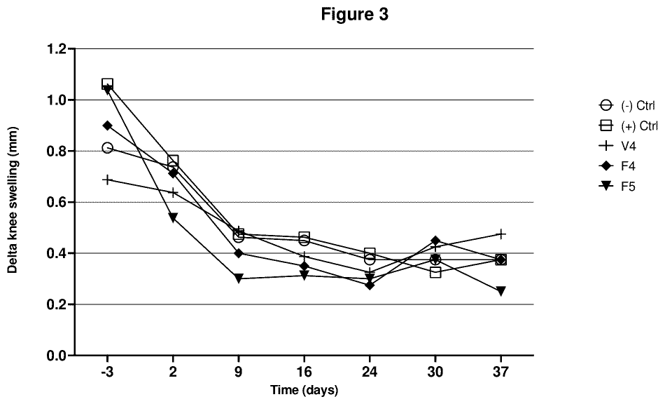

No change in rat general status was observed during the treatment, neither death nor significant body weight loss, indicating that the test items were well tolerated. Their clinical status remained good for all animals and no severe knee swelling was observed as illustrated in Table 4 and FIG. 3. Table 4 and FIG. 3 show the change in knee swelling in mm over time for rats administered the negative control, positive control, or formulations V4, F4 or F5.

TABLE 4

| Time | | | | | |
|---|---|---|---|---|---|
| (days) | (−) Ctrl | (+) Ctrl | V4 | F4 | F5 |
| −3 | 0.8125 | 1.0625 | 0.6875 | 0.9000 | 1.0375 |
| 2 | 0.7375 | 0.7625 | 0.6375 | 0.7125 | 0.5375 |
| 9 | 0.4625 | 0.4750 | 0.4875 | 0.4000 | 0.3000 |
| 16 | 0.4500 | 0.4625 | 0.3875 | 0.3500 | 0.3125 |
| 24 | 0.3750 | 0.4000 | 0.3250 | 0.2750 | 0.3000 |
| 30 | 0.3750 | 0.3250 | 0.4250 | 0.4500 | 0.3750 |
| 37 | 0.3750 | 0.3750 | 0.4750 | 0.3750 | 0.2500 |

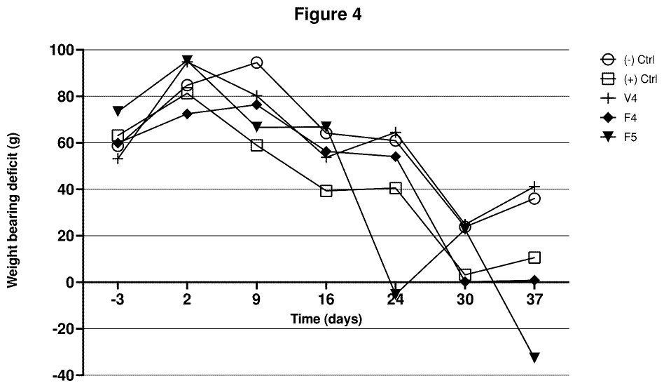

Results of weight bearing deficit over time expressed as the difference between the weight applied to the left hind paw and the weight applied to the right hind paw (in g) are presented in Table 5 and FIG. 4. A large positive value indicates that the animal is experiencing pain in its operated right leg. It can be observed that 37 days after treatment start, while results are similar for the negative control and V4, the delta weight is negative for F5 and close to zero for F4, suggesting that both doses are at least as effective as the positive control after 5 weeks.

TABLE 5

| Time | | | | | |
|---|---|---|---|---|---|
| (days) | (−) Ctrl | (+) Ctrl | V4 | F4 | F5 |
| −3 | 58.7500 | 63.1500 | 53.2500 | 59.9250 | 73.4000 |
| 2 | 84.8300 | 81.3875 | 94.8000 | 72.4750 | 95.3750 |
| 9 | 94.5500 | 58.9625 | 80.2500 | 76.4500 | 66.7250 |
| 16 | 64.2000 | 39.3500 | 53.7500 | 56.3750 | 66.8375 |
| 24 | 60.9500 | 40.5500 | 64.5500 | 54.1000 | −5.2500 |
| 30 | 23.8500 | 3.2500 | 24.9000 | 0.2000 | 22.7500 |
| 37 | 36.0500 | 10.6500 | 41.1500 | 0.9000 | −32.6000 |

Finally, histopathology analysis performed on toluidine blue stained knee-joint sample slides allowed an OA scoring ranking from 0 to 5 (0=normal tissue and 5=severe OA) established according to “the OARSI histopathology initiative—recommendations for histological assessments of osteoarthrosis in rat” Gerwin et al. 2010. Four expert histopathologists attributed a score, means and SD are given in table 6. Results show that the animals of the control group and vehicle V4 group had OA-affected right knees, while positive control IL-1Ra saline solution and both IL-1Ra formulations presented a reduced scoring with values close to each other suggesting a good efficacy of tested formulations. As an element of comparison, the non-operated left knees presented a score of 0 or 1 for all the animals.

TABLE 6

| | (−) Ctrl | (+) Ctrl | V4 | F4 | F5 |
|---|---|---|---|---|---|
| Left knee | 0.00 | 0.00 | 0.00 | 0.03 ± | 0.00 |
| | | | | 0.06 | |
| Right knee | 3.38 ± | 1.56 ± | 3.35 ± | 2.00 ± | 1.84 ± |
| | 0.87 | 0.63 | 0.39 | 0.35 | 0.37 |

### Example 5: IL1-Ra Formulations Stability Study

Protein stability within F4 and F5 formulations was assessed at study start and after 4 weeks of storage at 4° C. Study was completed with the assessment of F5 stability after 3 weeks of storage at 30° C. Formulations compositions are detailed in example 4.

Protein content and aggregation within formulations were measured according to the following protocol: Around 25 mg of formulation were accurately weighed into an Eppendorf which was then centrifuged 5 min at 13200 rpm. Supernatant (i.e. polymer vehicle) was removed using a pipette and pellet was washed three times with around 1 mL ethyl acetate to remove vehicle leftover. Pellet was dried for 1 hour in vacuum oven at 30° C. and then resuspended in 1 mL of SEC-Tween 80 buffer (50 mM NaP, pH:6.8; 100 mM NaCl; 0.2% NaN3; 0.005% (w/w) Tween 80). Diluted samples were analysed using an isocratic size-exclusion chromatography HPLC method carried out on a Waters W2690/5 instrument with a Xbridge™ BEH200A SEC 7.8×300 mm, 3.5 μm column, SEC buffer as eluent and a flow rate set at 1.0 mL/min. Protein content was quantified based on a calibration curve at 280 nm obtained with a Waters W2689 UV detector, and protein aggregation was followed using a Waters W2475 fluorescence detector with excitation and emission wavelengths set at 282 and 344 nm, respectively.

The analysis was performed in triplicate, mean results and SD are presented in table 7.

TABLE 7

| | | Study start | | 4 weeks at 4° C. | | 3 weeks at 30° C. | |
|---|---|---|---|---|---|---|---|
| | Theo. | Drug | | Drug | | Drug | |
| Test | content | content | Aggregation | content | Aggregation | content | Aggregation |
| item | % (w/w) | % (w/w) | (%) | % (w/w) | (%) | % (w/w) | (%) |
| F4 | 4.35 | 4.07 ± 0.02 | 2.08 ± 0.02 | 4.30 ± 0.23 | 2.14 ± 0.05 | NA | NA |
| F5 | 18.02 | 17.80 ± 2.05 | 2.05 ± 0.03 | 18.81 ± 0.06 | 2.23 ± 0.04 | 16.54 ± 0.65 | 2.14 ± 0.05 |

Results show that the protein aggregation is stable within formulations whatever the storage conditions and that the high molecular weight fraction does not exceed 2.5% of total protein content. After 4 weeks at 4° C., the measured protein contents are close to the theoretical ones, suggesting good protein stability. Under accelerated degradation, a light decrease in drug recovery is observed which can be mitigated with an accurate formulation storage.

### Example 6: Vehicles Bioresorption Study

The biocompatibility and the resorption of polymeric vehicles over time were evaluated after single intra articular injection into rat knees. Vehicle compositions are detailed in the following Table 8.

TABLE 8

| Test | | TB % | | DB % | Solvent | Solvent |
|---|---|---|---|---|---|---|
| item | TB type | (w/w) | DB type | (w/w) | type | % (w/w) |
| VA | P2R2 | 8 | dP2R2.4 | 32 | DMSO | 60 |
| VB | DL80G-P2R2 | 8 | DL80G-dP2R2.4 | 32 | DMSO | 60 |
| VC | P1R6 | 8 | dP1R4 | 32 | DMSO | 60 |
| VE | P3R2 | 12 | dP2R2.4 | 12 | Tripropionin | 76 |

Briefly, 20 μL of test items were slowly injected intra-articularly into the right knee of ten- to twelve-week-old male Wistar rats using a Hamilton syringe with a 25G needle. Daily clinical examination and a thorough weekly examination of all animals were done after administration of the tested compounds.

After 1, 21, 42 and 84 days, 6 animals per group of candidate vehicle were

euthanized. Before euthanasia, blood samples were withdrawn by cardiac puncture and the serum obtained by centrifugation was used to perform different ELISA tests to quantify biomarker levels.

After euthanasia both knee joints samples were collected. At each time-point, for half of the animals, the samples were fixed in 4% formalin for 2 days, followed by decalcification based on EDTA disodium 20% and embedded in paraffin blocks. Sections of 4 to 8 mm thick were further stained with H&E (hematoxylin and eosin) and toluidine blue and scanned by slide scanner. Additionally, PEG immunohistochemistry (IHC) staining was also performed using rabbit monoclonal antibody to conjugated PEG (Abcam ref Ab51257), rabbit monoclonal lgG as an isotype control (Abcam ref Ab172730), goat anti-rabbit IgG H&L (Abcam ref Ab205718) and DAB substrate kit (Abcam ref Ab64238). Three expert physiopathologists attributed to each knee joint an inflammation score ranking from 0 to 5 (0=normal tissue and 5=severe OA) and a total joint score ranking from 0 to 30 established according to “the OARSI histopathology initiative—recommendations for histological assessments of osteoarthrosis in rat” Gerwin et al. 2010. Similarly, a PEG histology score ranging from 0 to 5 (0=absence of PEG and 5=very high presence of PEG) was attributed for each knee sample.

The remaining knee samples were dissected, solubilized overnight in 7 mL of 5M NaOH at 50° C. and centrifuged for 5 min at 3000 rpm. A known volume of supernatant was collected and then neutralized with 5 mL of 5N HCl. The obtained hydrolysed samples were then diluted and the amount of D-Lactate in each sample was quantified using a D-Lactic acid kit purchased from Megazyme (Product code K-DATE).

### Biocompatibility Results:

Regarding the clinical aspects, all of the animals, at each time-point of the study, exhibited normal behaviours, body weight, water, and food consumption. Biological analyses were in accordance with the clinical observations and did not reveal any detectable systemic toxicity signs.

Histopathological changes when observed in right knee sections were characteristic of rather minimal to mild and limited superficial lesions of the joint cartilage and lateral synovial membrane. All cartilage lesions were considered at limit of detection and could be observed as incidental findings in untreated control animals. No remarkable differences in incidence, severity and extent of cartilage histopathological changes could be observed between groups. A good biocompatibility was thus observed with the 4 polymeric vehicles, whatever the copolymers and/or solvent used.

### Polymer Resorption Results:

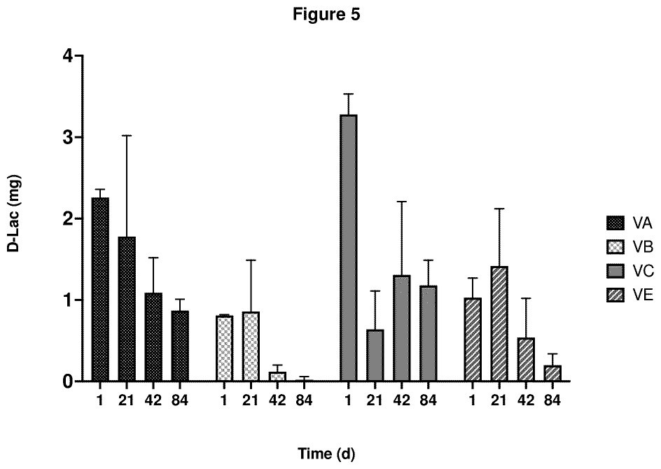

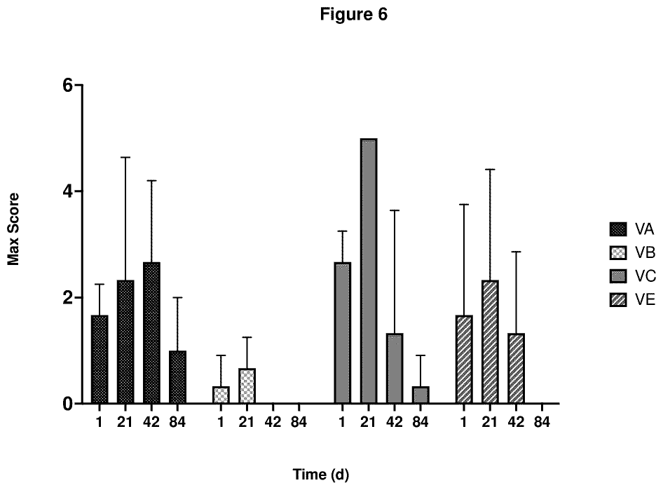

Results of D-Lactic acid quantification and PEG IHC scoring are presented in FIGS. 5 and 6 respectively.

For all test items a decreased level of lactic acid was measured at the end of the study, confirming the degradation of the polyester within the intraarticular area. The resorption of the copolymer was further confirmed by the evolution of the IHC scoring highlighting a lower level of PEG after 84 days for all tested vehicles. Interestingly, depending on the composition, different results are obtained for both lactic acid quantification and PEG IHC. In particular with VB, almost no polymer was detected after 42 days, indicating that the time of depot resorption is highly linked to the copolymer type. The incorporation of a 20% molar fraction of glycolide within the polyester arms can cause a large decrease in the time period required for polymer degradation within the intra-articular joint space, which would be beneficial if a short release duration for the API and/or repeated administrations of the API are required.

1. A pharmaceutical composition comprising
(a) a triblock copolymer having the formula:
Aᵥ-B_{w}-Aₓ
wherein A is a polyester, B is polyethylene glycol, v and x are numbers of repeat units ranging from 1 to 3,000 and w is a number of repeat units ranging from 3 to 300 and v=x or v≠x in an amount of from about 3 w/w % to 25 w/w % of the total composition;
(b) a diblock copolymer having the formula:
C_{y}-A_{z}
wherein A is a polyester, C is an end-capped polyethylene glycol, and y and z are numbers of repeat units with y ranging from 2 to 250 and z ranging from 1 to 3,000 in an amount of from about 3 w/w % to 35 w/w % of the total composition;
(c) a therapeutic protein which is an Interleukin-1 antagonist in an amount of from about 0.5 w/w % to 25 w/w % of the total composition;
(d) optionally one or more stabilizer compounds in an amount of from about 0.25 w/w % to 15 w/w % of the total composition; and
(e) organic solvent in an amount of from about 50 w/w % to 80 w/w % of the total composition.

2. A pharmaceutical composition according to claim 1 wherein for the triblock copolymer w is an integer from about 20 to 75 and v and x are each an integer from about 35 to 85; and/or wherein for the triblock copolymer a molecular weight of the PEG is from about 1 kDa to 3 kDa and a polyester repeat unit to ethylene oxide molar ratio is from about 2 to 6.

3. A pharmaceutical composition according to claim 1 wherein for the triblock copolymer w is an integer from about 20 to 25 and v and x are each an integer from about 40 to 85, preferably wherein w is about 23 and v and x are each about 68 or wherein w is an integer from about 40 to 50 and v and x are each an integer from about 35 to 75, preferably wherein w is about 45 and v and x are each about 45 or wherein w is an integer from about 60 to 75 and v and x are each an integer from about 55 to 85, preferably wherein w is about 68 and v and x are each about 68; and/or wherein for the triblock copolymer the molecular weight of the PEG is about 3 kDa and the polyester repeat unit to ethylene oxide molar ratio is about 2; or the molecular weight of the PEG is about 1 kDa and a polyester repeat unit to ethylene oxide molar ratio is about 6 or the molecular weight of the PEG is about 2 kDa and the polyester repeat unit to ethylene oxide molar ratio is about 2.

4. A composition according to claim 1 wherein for the diblock copolymer y is an integer from about 20 to 50 and z is an integer from about 75 to 150; and/or wherein for the diblock copolymer a molecular weight of the PEG is from about 1 to 2 kDa and a polyester repeat unit to ethylene oxide molar ratio is from about 2 to 4.

5. A composition according to claim 1 wherein for the diblock copolymer y is an integer from about 20 to 25 and z is an integer from about 75 to 110, preferably wherein y is about 23 and z is about 90; or wherein y is an integer from about 40 to 50 and z is an integer from about 100 to 150, preferably wherein y is about 45 and z is about 136; and/or wherein for the diblock copolymer a molecular weight of the PEG is about 1 kDa and a polyester repeat unit to ethylene oxide molar ratio is about 4; or the molecular weight of the PEG is about 2 kDa and the polyester repeat unit to ethylene oxide molar ratio is about 2 or about 3.

6. A composition according to claim 1 wherein the polyester A for the diblock copolymer is selected from the group of poly(lactic acid), poly(lactic-co-glycolic acid), polyglycolic acid, polycaprolactone, poly(ε-caprolactone-co-lactide), polyethylene adipate, polydioxanone, polyhydroxyalkanoate, and mixtures thereof.

7. A composition according to claim 1 wherein each polyester A for the triblock copolymer is selected from the group of poly(lactic acid), poly(lactic-co-glycolic acid), polyglycolic acid, polycaprolactone, poly(ε-caprolactone-co-lactide), polyethylene adipate, polydioxanone, polyhydroxyalkanoate, and mixtures thereof.

8. A composition according to claim 1 wherein the polyester A for the diblock copolymer comprises poly(lactic acid) or poly(D,L-lactic acid), or preferably is poly(D,L-lactic-co-glycolic acid) or poly(D,L-lactic acid).

9. A composition according to claim 1 wherein each polyester A for the triblock copolymer comprises poly(lactic acid) or poly(D,L-lactid acid), or preferably is poly(D,L-lactic-co-glycolic acid) or poly(D,L-lactic acid).

10. A composition according to claim 1 wherein the poly(D,L-lactic-co-glycolic acid) comprises at least 60% (mol/mol) lactic acid, optionally at least 80% (mol/mol) lactic acid.

11. A composition according to claim 1 wherein the therapeutic protein is IL-1Ra, or an amino acid sequence having at least 80%, optionally at least 85%, optionally at least 90%, optionally at least 95%, optionally at least 99% sequence identity to the amino acid sequence according to SEQ ID NO: 1, optionally wherein the therapeutic protein consists of the amino acid sequence according to SEQ ID NO: 1.

12. A composition according to claim 1 wherein the therapeutic protein is canakinumab, optionally an antibody wherein each heavy chain has at least 80%, optionally at least 85%, optionally at least 90%, optionally at least 95%, optionally at least 99% sequence identity to the amino acid sequence according to SEQ ID NO:
2, optionally consisting of SEQ ID NO: 2; and each light chain has at least 80%, optionally at least 85%, optionally at least 90%, optionally at least 95%, optionally at least 99% sequence identity to the amino acid sequence according to SEQ ID NO: 3, optionally consisting of SEQ ID NO: 3.

13. A composition according to claim 1 wherein the therapeutic protein is rilonacept, or an amino acid sequence having at least 80%, optionally at least 85%, optionally at least 90%, optionally at least 95%, optionally at least 99% sequence identity to the amino acid sequence according to SEQ ID NO: 4, optionally wherein the therapeutic protein consists of the amino acid sequence according to SEQ ID NO: 4.

14. A composition according to claim 1 wherein the therapeutic protein is a single domain antibody.

15. A composition according to claim 1 wherein the therapeutic protein has a molecular weight of from about 10 kDa to about 260 kDa, optionally about 10 to about 150 kDa, optionally about 10 to about 146 kDa, optionally about 10 to about 140 kDa, optionally about 10 kDa to about 100 kDa, optionally about 10 kDa to about 50 kDa, optionally about 10 kDa to about 30 kDa, optionally about 15 to about 20 kDa.

16. A composition according to claim 1 comprising from about 3 w/w % to 20 w/w % of the therapeutic protein, optionally from about 4 w/w % to 16 w/w % of the therapeutic protein.

17. A composition according to claim 1 comprising from about 60 w/w % to 80 w/w %, optionally from about 65 w/w % to 80 w/w %, preferably from about 65 w/w % to 75 w/w % organic solvent.

18. A composition according to claim 1 wherein the organic solvent is selected from benzyl alcohol, benzyl benzoate, dimethyl isosorbide (DMI), ethyl acetate, ethyl benzoate, ethyl lactate, glycerol formal, methyl ethyl ketone, methyl isobutyl ketone, N-ethyl-2-pyrrolidone, pyrrolidone-2, tetraglycol, triacetin, tributyrin, tripropionin, glycofurol, and mixtures thereof.

19. A composition according to claim 18 wherein the organic solvent is tripropionin or benzyl benzoate.

20. A composition according to claim 1 comprising about 1.5 w/w % to 10 w/w % stabilizer compound, optionally about 4 w/w % to 8 w/w % stabilizer compound, optionally about 5 w/w % stabilizer compound.

21. A composition according to claim 1 wherein the stabilizer compound is trehalose, L-methionine, sucrose, mannitol, ascorbic acid, arginine, polysorbate 80, polysorbate 20 or a combination thereof, preferably trehalose, L-methionine, or a combination thereof.

22. A composition according to claim 1 comprising from about 3 w/w % to about 20 w/w %, optionally from about 3 w/w % to about 15 w/w %, optionally from about 3 w/w % to 10 w/w %, optionally from about 9 w/w % of the triblock copolymer.

23. A composition according to claim 1 comprising from about 3 w/w % to about 25 w/w %, optionally from about 3 w/w % to about 20 w/w %, optionally from about 3 w/w % to about 15 w/w %, optionally from about 3 w/w % to 10 w/w %, optionally from about 9 w/w % of the diblock copolymer.

24. A composition according to claim 1 comprising less than about 50 w/w % total copolymer, preferably less than about 40 w/w % total copolymer.

25. A composition according to claim 1 further comprising one or more salts and/or one or more buffering agents, optionally wherein the buffering agent is sodium phosphate or histidine.

26. A composition according to claim 1 for use in medicine.

27. A composition according to claim 1 for use in preventing, treating, or ameliorating a rheumatic disease in a subject.

28. A composition for use according to claim 27 wherein the rheumatic disease is selected from osteoarthritis, crystal arthritis, post-traumatic osteoarthritis, rheumatoid arthritis, ankylosing spondylitis, fibromyalgia, infectious arthritis, juvenile idiopathic arthritis, lupus erythematosus, polymyalgia rheumatica, psoriatic arthritis, reactive arthritis, post-traumatic osteoarthritis, and scleroderma, preferably osteoarthritis, post-traumatic osteoarthritis or crystal arthritis.

29. A composition for use according to claim 26 wherein the use comprises administering the composition to the subject by intra-articular injection or peri-articular injection.

30. A composition for use according to claim 29 wherein the composition is injected into or in the vicinity of a synovial joint such as a knee, ankle, elbow, humerus, ulna, pivot joint, ball and socket joint, hinge joint, shoulder, hip, scapula, leg joint, fibula, saddle joint, wrist joint, finger joint, toe joint or tibia of the subject, preferably the knee, toe, shoulder or hip.

31. A composition according to claim 1 which is suitable for forming a depot when injected into the body.

32. A method of preventing, treating, or ameliorating rheumatic disease in a subject, the method comprising the step of administering a composition as defined in claim 1 to the subject.

33. A method according to claim 32 wherein the rheumatic disease is selected from osteoarthritis, crystal arthritis, post-traumatic osteoarthritis, rheumatoid arthritis, ankylosing spondylitis, fibromyalgia, infectious arthritis, juvenile idiopathic arthritis, lupus erythematosus, polymyalgia rheumatica, psoriatic arthritis, reactive arthritis, and scleroderma, preferably osteoarthritis, post-traumatic osteoarthritis or crystal arthritis.

34. A method according to claim 32 wherein the composition is administered to the subject by intra-articular injection or peri-articular injection.

35. A method according to claim 34 wherein the composition is injected into or in the vicinity of a synovial joint such as a knee, ankle, elbow, humerus, ulna, pivot joint, ball and socket joint, hinge joint, shoulder, hip, scapula, leg joint, fibula, saddle joint, wrist joint, finger joint, toe joint, or tibia of the subject, preferably the knee, toe, shoulder or hip.

36. A method according to claim 32 wherein the composition is administered using an 18G to 26G needle, optionally a 21G to 23G needle.

37. A method according to claim 32 wherein a ratio of a concentration of the therapeutic protein in synovial fluid at an administration site to a concentration of the therapeutic protein in serum of the subject is greater than 10, optionally greater than 50, optionally greater than 100 for a period of at least 7 days, optionally 14 days, optionally 21 days, optionally 28 days, optionally 1 month, optionally 2 months, optionally 3 months after administration of the composition.

38. A method of preparing a pharmaceutical composition of claim 1, the method comprising
a) preparing an aqueous solution of the therapeutic protein as;
b) optionally adding the stabilizer compound to the solution;
c) spray drying the solution containing the stabilizer compound of step b) to form a spray-dried product;
d) dissolving the triblock copolymer and the diblock copolymer in the organic solvent;
e) adding the spray-dried product to the polymer composition formed in step d) and mixing the composition to form a dispersion of the therapeutic protein.

39. A method according to claim 38 wherein respective amounts of triblock and diblock copolymers dissolved in step d) are sufficient to provide a final dispersion in step e) comprising the triblock copolymer in an amount of from 3 w/w % to 25 w/w % of the final composition and the diblock copolymer in an amount of from 3 w/w % to 25 w/w %.

40. A method according to claim 38 wherein the amount of therapeutic protein in the dispersion of step e) is from 0.5 w/w % to 25 w/w %, optionally from about 3 w/w % to 20 w/w %, optionally from about 4 w/w % to 16 w/w %.

41. A method according to claim 38 wherein an amount of stabilizer compound in the final dispersion is from about 0.25 w/w % to 15 w/w %, optionally about 1.5 w/w % to 10 w/w % stabilizer compound, optionally about 4 w/w % to 8 w/w % stabilizer compound, optionally about 5 w/w %.

42. A method according to claim 38 wherein the aqueous solution of step a) comprises one or more salts and/or one or more buffering agents.

43. A method according to claim 42 wherein the aqueous solution comprises sodium phosphate or histidine.

44. A method according to claim 38 wherein the aqueous solution comprises polysorbate 80.

45. A method according to claim 38 wherein the product of step b) is filtered prior to spray drying.
